(19) United States
(12) Patent Application Publication
(10) Pub. No.: US 2015/0038735 A1
(43) Pub. Date: Feb. 5, 2015

(54) PREPARATION OF ALPHA, BETA-UNSATURATED CARBOXYLIC ACIDS AND ESTERS THEREOF

(71) Applicant: MYRIANT CORPORATION, Quincy, MA (US)

(72) Inventors: Cenan A. Ozmeral, Boston, MA (US); Joseph P. Glas, Shelton, SC (US); Rajesh Dasari, Lincoln, MA (US); Setrak Tanielyan, Maplewood, NJ (US); Ramesh Deoram Bhagat, Avenel, NJ (US); Mohan Reddy Kasireddy, Avenel, NJ (US); Ramnik Singh, Winchester, MA (US); Vijay Gnanadesikan, Stoneham, MA (US); Robert L. Augustine, Livingston, NJ (US); Santosh More, Newark, NJ (US)

(21) Appl. No.: 14/383,553

(22) PCT Filed: Sep. 6, 2013
(86) PCT No.: PCT/US2013/029368

§ 371 Date: Sep. 6, 2014

Related U.S. Application Data
(60) Provisional application No. 61/608,053, filed on Mar. 7, 2012.
(60) Provisional application No. 61/402,913, filed on Sep. 7, 2010.

(51) Int. Cl. C07C 67/327; C07C 51/377

(52) U.S. Cl. CPC C07C 67/327; C07C 51/377
USPC 560/212; 562/599; 562/606

(57) ABSTRACT

An L-type zeolite, a modified L-type zeolite, or any combination thereof may be useful in catalytically preparing α,β-unsaturated carboxylic acids and/or esters thereof through reaction pathways that include dehydroxylation reactions and optionally esterification reactions. In some reaction pathways, dehydroxylation reactions and esterification reactions may be performed sequentially or concurrently.

### CROSS-REFERENCE TO RELATED APPLICATION

This application claims the priority of the U.S. Provisional Application Ser. No. 61/608,053, filed on Mar. 7, 2012; U.S. Provisional Application Ser. No. 61/740,230, filed on Dec. 20, 2012; and U.S. application Ser. No. 13/819,035, filed Feb. 26, 2013.

### BACKGROUND

The present invention relates to methods for catalytically preparing α,β-unsaturated carboxylic acids and/or esters thereof.

Acrylic acid, and its ester derivatives, is an important commercial chemical used in the production of polyacrylic esters, elastomers, superabsorbent polymers, floor polishes, adhesives, paints, and the like. Historically, acrylic acid has been produced by hydroxycarboxylation of acetylene. This method utilizes nickel carbonyl and high pressure carbon monoxide, both of which are expensive and considered environmentally unfriendly. Other methods, e.g., those utilizing ethenone and ethylene cyanohydrin, generally have the same pitfalls.

In an effort to, inter alia, decrease environmental impact, lactic acid dehydroxylation has been investigated as a route to produce acrylic acid because lactic acid can be derived from renewable, biological resources like sugar cane. Predominantly, solid catalysts have been investigated for use in both liquid phase and vapor phase dehydroxylation reactions that convert lactic acid to acrylic acid. Specific solid catalysts that have been investigated include sodium phosphates supported on silica and weak acids supported on aluminosilicates or silica. Dehydroxylation reactions performed with some of these catalysts have been shown to proceed only at higher temperatures in excess of 350° C., which may add significant energy costs if scaled-up. Further, many solid catalysts, including those above, have shown poor selectivity to acrylic acid (i.e., a plurality of byproducts that need to be removed, which increases manufacturing costs) and a low overall yield of the reaction. Accordingly, the cost to manufacture acrylic acid via lactic acid dehydroxylation is still very high.

### SUMMARY OF THE INVENTION

The present invention relates to methods for catalytically preparing α,β-unsaturated carboxylic acids and/or esters thereof.

One embodiment of the present invention provides for a method that comprises: providing a composition comprising a reactant selected from the group consisting of an α-hydroxycarboxylic acid, an α-hydroxycarboxylic acid ester, a β-hydroxycarboxylic acid, a β-hydroxycarboxylic acid ester, an α-alkoxycarboxylic acid, an α-alkoxycarboxylic acid ester, a β-alkoxycarboxylic acid, a β-alkoxycarboxylic acid ester, a lactide, and any combination thereof and performing a dehydroxylation reaction by contacting the composition with a dehydroxylation catalyst, thereby producing a product comprising an α,β-unsaturated carboxylic acid and/or ester thereof, the dehydroxylation catalyst comprising at least one selected from the group consisting of an L-type zeolite, a modified L-type zeolite, and any combination thereof.

Another embodiment of the present invention provides for a method that comprises: providing a composition comprising a reactant selected from the group consisting of an α-hydroxycarboxylic acid, an α-hydroxycarboxylic acid ester, a β-hydroxycarboxylic acid, a β-hydroxycarboxylic acid ester, an α-alkoxycarboxylic acid, an α-alkoxycarboxylic acid ester, a β-alkoxycarboxylic acid, a β-alkoxycarboxylic acid ester, a lactide, and any combination thereof and concurrently performing an esterification reaction and a dehydroxylation reaction by contacting the composition with an alcohol and a catalyst, thereby yielding a product that comprises an α,β-unsaturated carboxylic acid ester, the catalysts comprising at least one selected from the group consisting of an L-type zeolite, a modified L-type zeolite, and any combination thereof.

Yet another embodiment of the present invention provides for a method that comprises: providing a composition comprising a reactant selected from the group consisting of an α-hydroxycarboxylic acid, a β-hydroxycarboxylic acid, an α-alkoxycarboxylic acid, a β-alkoxycarboxylic acid, and any combination thereof; performing an esterification reaction by contacting the composition with an esterification catalyst and an alcohol, thereby producing an intermediate comprising an ester of the reactant; then performing a dehydroxylation reaction by contacting intermediate with a dehydroxylation catalyst, thereby producing a product comprising an α,β-unsaturated carboxylic acid ester, the dehydroxylation catalyst comprising at least one selected from the group consisting of an L-type zeolite, a modified L-type zeolite, and any combination thereof.

Another embodiment of the present invention provides for a method that comprises: providing a composition comprising a reactant selected from the group consisting of an α-hydroxycarboxylic acid, a β-hydroxycarboxylic acid, an α-alkoxycarboxylic acid, a β-alkoxycarboxylic acid, and any combination thereof; performing an esterification reaction by contacting the composition with an alcohol, thereby producing an intermediate comprising an ester of the reactant, wherein the esterification reaction is carried out with an exogenous catalyst; and then performing a dehydroxylation reaction by contacting intermediate with a dehydroxylation catalyst, thereby producing a product comprising an α,β-unsaturated carboxylic acid ester, the dehydroxylation catalyst comprising at least one selected from the group consisting of an L-type zeolite, a modified L-type zeolite, and any combination thereof.

Yet another embodiment of the present invention provides for a method that comprises: providing a composition comprising a reactant selected from the group consisting of an α-hydroxycarboxylic acid, an α-hydroxycarboxylic acid ester, a β-hydroxycarboxylic acid, a β-hydroxycarboxylic acid ester, an α-alkoxycarboxylic acid, an α-alkoxycarboxylic acid ester, a β-alkoxycarboxylic acid, a β-alkoxycarboxylic acid ester, a lactide, and any combination thereof; and performing a dehydroxylation reaction in the presence of a carrier gas by contacting the composition with a dehydroxylation catalyst, thereby producing a product comprising an α,β-unsaturated carboxylic acid and/or ester thereof, the carrier gas comprising greater than about 90% carbon dioxide.

Another embodiments of the present invention provides for a method that comprises: producing acrylic acid or acrylic acid ester from a reactant derived via a fermentation process involving a biological catalyst and a biological source, the biological source comprising at least one of glucose, sucrose, glycerol, and any combination thereof, and the reactant comprising selected from the group consisting of an α-hydroxycarboxylic acid, an α-hydroxycarboxylic acid ester, a β-hydroxycarboxylic acid, a β-hydroxycarboxylic acid ester, an α-alkoxcarboxylic acid, an α-alkoxycarboxylic acid ester, a β-alkoxycarboxylic acid, a β-alkoxycarboxylic acid ester, a lactide, and any combination thereof.

Yet another embodiments of the present invention provides for a method that comprises: producing acrylic acid or acrylic acid ester from a reactant derived via a chemical process involving a chemical catalyst and a biological source, the biological source comprising at least one of glucose, sucrose, glycerol, and any combination thereof, and the reactant comprising selected from the group consisting of an α-hydroxycarboxylic acid, an α-hydroxycarboxylic acid ester, a β-hydroxycarboxylic acid, a β-hydroxycarboxylic acid ester, an α-alkoxcarboxylic acid, an α-alkoxycarboxylic acid ester, a β-alkoxycarboxylic acid, a β-alkoxycarboxylic acid ester, a lactide, and any combination thereof.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates nonlimiting examples of several different reaction pathways described herein.

FIG. 2 provides an illustrative system schematic for use in preparing α,β-unsaturated carboxylic acids and/or esters thereof according to at least some embodiments of the present invention.

FIG. 3 provides the chemical pathway for the conversion of triglyceride to acrylic acid using chemical catalysts.

FIG. 4 provides the chemical pathway for the conversion of glucose to acrylic acid using chemical catalysts.

FIG. 5 provides the conversion percentage and selectivity of a reaction performed with an L-type zeolite according to at least some embodiments of the present invention.

FIG. 6 provides the conversion percentage and selectivity of a reaction performed with an L-type zeolite and a regenerated L-type zeolite according to at least some embodiments of the present invention.

FIG. 7 provides the conversion percentage and selectivity of a reaction performed with an L-type zeolite according to at least some embodiments of the present invention.

### DETAILED DESCRIPTION

The present invention relates to methods for catalytically preparing α,β-unsaturated carboxylic acids and/or esters thereof.

The present invention provides for, in at least some embodiments, reaction pathways utilizing L-type zeolite catalysts that effectively (i.e., with higher conversion percentages) and selectively produce α,β-unsaturated carboxylic acids (e.g., acrylic acid) and/or esters thereof from lactic acid-like reactants. As illustrated further herein, surprisingly, L-type catalysts have been shown, in some embodiments, to more effectively and selectively produce α,β-unsaturated carboxylic acids (e.g., acrylic acid) and/or esters thereof from lactic acid-like reactants. Consequently, reaction pathways and catalysts described herein may, in some embodiments, provide for cost-effective, environmentally friendly industrial scale production of acrylic acid from lactic acid.

It should be noted that when “about” is used herein at the beginning of a numerical list, “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

As used herein, the term “reaction pathway” refers to the reaction or series of reactions for converting reactants to products that comprise an α,β-unsaturated carboxylic acid or the ester thereof, where intermediates are optionally formed in the reaction or series of reactions. In some embodiments, a reaction pathway of the present invention may comprise a dehydroxylation reaction utilizing dehydroxylation catalysts that comprise L-type zeolites. In some embodiments, a reaction pathway of the present invention may further comprise an esterification reaction.

As used herein, the term “dehydroxylation reaction” refers to the removal of water from a reactant. The term “dehydroxylation reaction” is also known as “dehydration reaction” in the art.

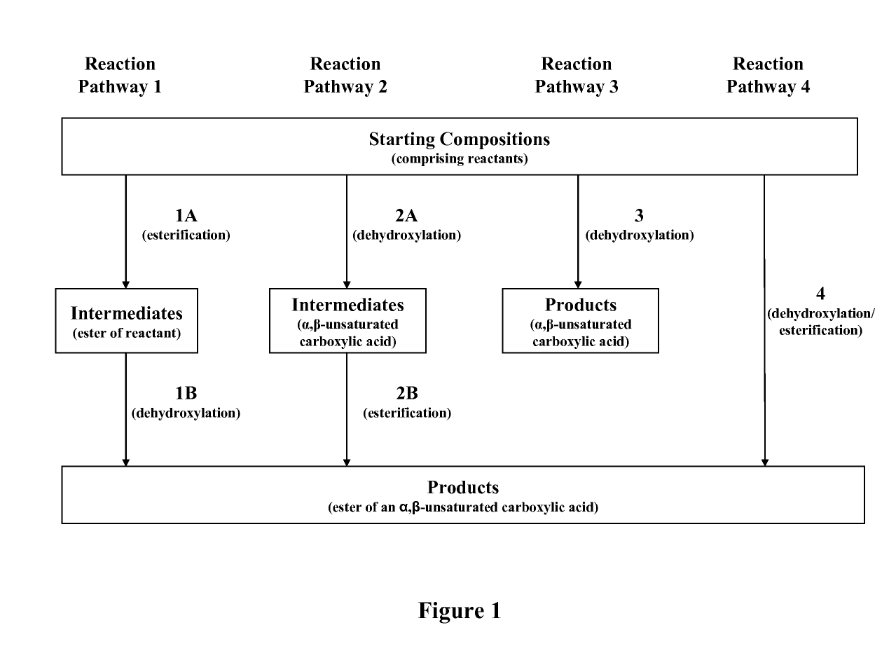

FIG. 1 illustrates nonlimiting examples of several different reaction pathways of the present invention. For example, in some embodiments as shown in Reaction Pathway 1 of FIG. 1, beginning with a starting composition that comprises reactants, a reaction pathway of the present invention may comprise an esterification reaction (1A) that yields an intermediate that comprises an ester of the reactant followed by a dehydroxylation reaction (1B) that yields a product that comprises an ester of an α,β-unsaturated carboxylic acid. In other embodiments as shown in Reaction Pathway 2 of FIG. 1, beginning with a starting composition that comprises reactants, a reaction pathway of the present invention may comprise a dehydroxylation reaction (2A) that yields an intermediate that comprises an α,β-unsaturated carboxylic acid followed by an esterification reaction (2B) that yields a product that comprises an ester of the α,β-unsaturated carboxylic acid. In yet other embodiments as shown in Reaction Pathway 3 of FIG. 1, beginning with a starting composition that comprises reactants, a reaction pathway of the present invention may comprise a dehydroxylation reaction (3) that yields a product that comprises an α,β-unsaturated carboxylic acid. In other embodiments as shown in Reaction Pathway 4 of FIG. 1, beginning with a starting composition that comprises reactants, a reaction pathway of the present invention may comprise a concurrent dehydroxylation and esterification reaction (4) that yields a product that comprises an ester of the α,β-unsaturated carboxylic acid.

Reactants suitable for use in conjunction with reaction pathways of the present invention may include, but are not limited to, an α-hydroxycarboxylic acid, an α-hydroxycarboxylic acid ester, a β-hydroxycarboxylic acid, a β-hydroxycarboxylic acid ester, an α-alkoxycarboxylic acid, an α-alkoxycarboxylic acid ester, a β-alkoxycarboxylic acid, a β-alkoxycarboxylic acid ester, and the like, and any combination thereof. Suitable aforementioned esters may be C₁-C₁₀ alkyl esters. Specific examples of reactants may include, but are not limited to, lactic acid, salts of lactic acid (e.g., calcium, ammonium, magnesium, sodium, and potassium salts thereof), an alkyl ester of lactic acid, lactide, methyl lactate, butyl lactate, 3-hydroxypropionic acid, an alkyl ester of 3-hydroxypropionic acid, methyl 3-hydroxypropionate, butyl 3-hydroxypropionate, lactamide, and any combination thereof. In some embodiments, reactants may be in the form of solids, liquids, melts, or gases. Further, reactants may be substantially pure chiral reactants or a racemic mixture of chiral reactants, e.g., D(−) lactic acid, L(+) lactic acid, DD lactide, LL lactide, D/L racemic mixtures of lactic acid, or D/L racemic mixtures of lactide.

Reactants suitable for use in conjunction with reaction pathways of the present invention may be produced by any known means. In some embodiments, reactants may be biologically-derived, chemically-derived, or a combination thereof. Examples of biologically-derived reactants may be found in International Patent Application No. PCT/US11/50707 entitled “Catalytic Dehydroxylation of Lactic Acid and Lactic Acid Esters,” the entirety of which is incorporated herein by reference. By way of nonlimiting example, lactic acid may be derived from the lactic acid salts (e.g., ammonium lactate) present in a fermentation broth produced from microorganisms (e.g., acid-tolerant homolactic acid bacteria) that utilize and/or metabolize sucrose, glucose, and the like from sugar cane, beets, whey, and the like.

In some embodiments, an α-hydroxy carboxylic acid described herein (e.g., lactic acid and its derivatives) may be obtained from a fermentation broth. In some embodiments, a fermentation broth described herein may be derived from the cultures of the bacterial species including Escherichia coli and Bacillus coagulans selected for lactic acid production on a commercial scale. In some embodiments, a fermentation broth described herein may be derived from the culture fluid of the filamentous fungal species selected for lactic acid production. In some embodiments, a fermentation broth described herein may be derived from yeast species known to produce lactic acid in industrial scale. Microorganisms suitable for the production of lactic acid on a commercial scale may, in some embodiments, include Escherichia coli, Bacillus coagulans, Lactobacillus delbruckii, L. bulgaricus, L. thermophilus, L. leichmanni, L. casei, L. fermentii, Streptococcus thermophilus, S. lactis, S. faecalils, Pediococcus sp, Leuconostoc sp, Bifidobacterium sp, Rhizopus oryzae and a number of species of yeasts in industrial use. One skilled in the art with the benefit of this disclosure should recognize suitable combinations of any of the foregoing.

The fermentation process for producing α-hydroxy carboxylic acid like lactic acid may, in some embodiments, be a batch process, a continuous process, or a hybrid thereof. A large number of carbohydrate materials derived from natural resources can be used as a feedstock in conjunction with the fermentative production of α-hydroxy carboxylic acids described herein. For example, sucrose from cane and beet, glucose, whey containing lactose, maltose and dextrose from hydrolyzed starch, glycerol from biodiesel industry, and combinations thereof may be suitable for the fermentative production of α-hydroxy carboxylic acids described herein. Microorganisms may also be created with the ability to use pentose sugars derived from hydrolysis of cellulosic biomass in the production of α-hydroxy carboxylic acids described herein. In some embodiments, a microorganism with ability to utilize both 6-carbon containing sugars such as glucose and 5-carbon containing sugars such as xylose simultaneously in the production of lactic acid is a preferred biocatalyst in the fermentative production of lactic acid. In some embodiments, hydrolysate derived from cheaply available cellulosic material contains both C-5 carbon and C-6 carbon containing sugars and a biocatalyst capable of utilizing simultaneously C-5 and C-6 carbon containing sugars in the production of lactic acid is highly preferred from the point of producing low-cost lactic acid suitable for the conversion into acrylic acid and acrylic acid ester.

In some embodiments, fermentation broths for the production of lactic acid may include acid-tolerant homolactic acid bacteria. By “homolactic” it is meant that the bacteria strain produces substantially only lactic acid as the fermentation product. The acid-tolerant homolactic bacteria is typically isolated from the corn steep water of a commercial corn milling facility. An acid tolerant microorganism, which can also grow at elevated temperatures, may be preferred in some embodiments. In some preferred embodiments, microorganisms that can produce at least 4 g of lactic acid per liter (and more preferably 50 g of lactic acid per liter) of the fermentation fluid may be utilized in fermentation procedures described herein.

In some embodiments, the fermentation broth may be utilized at various points of production, e.g., after various unit operations have occurred like filtration, acidification, polishing, concentration, or having been processed by more than one of the aforementioned unit operations. In some embodiments, when the fermentation broth may contain about 6 to about 20% lactic acid on weight/weight (w/w) basis, the lactic acid may be recovered in a concentrated form. The recovery of lactic acid in a concentrated form from a fermentation broth may be achieved by a plurality of methods and/or a combination of methods known in the art.

During the fermentation methods described herein, at least one alkali material (e.g., NaOH, CaCO₃, (NH₄)₂CO₃. NH₄HCO₃NH₄OH, KOH, or any combination thereof) may be utilized in order to maintain the near neutral pH of the growth medium. Addition of alkali materials to the fermentation broth often results in the accumulation of lactic acid in the form of inorganic salts. In some embodiments, ammonium hydroxide may be a preferred alkali material for maintaining the neutral pH of the fermentation broth. With the addition of ammonium hydroxide to the fermentation medium, ammonium lactate may accumulate in the fermentation broth. Because ammonium lactate has higher solubility in aqueous solution, it may have an increased concentration in the fermentation broth. One way to obtain lactic acid from the fermentation broth containing ammonium lactate may include micro and ultra filtrations of the fermentation broth followed by continuous ion exchange (CIX), simulated moving bed chromatography (SMB), electrodialysis bipolar membrane (EDBM), fixed bed ion exchange, or liquid-liquid extraction. The sample coming out of fixed bed ion exchange may, in some embodiments, then be subjected to bipolar electrodialysis to obtain lactic acid in the form of a concentrated free acid.

In some embodiments, the reactants (e.g., lactic acid and lactic acid ester) may be derived from biological resources (e.g., glucose, sucrose and glycerol) through one or more chemical processes using chemical catalysts without involving any fermentation process using biocatalysts. For example, lactic and lactic acid esters derived from the biological resources may be subsequently subjected to dehydroxylation and esterification reactions as described above to yield acrylic acid and acrylic acid ester.

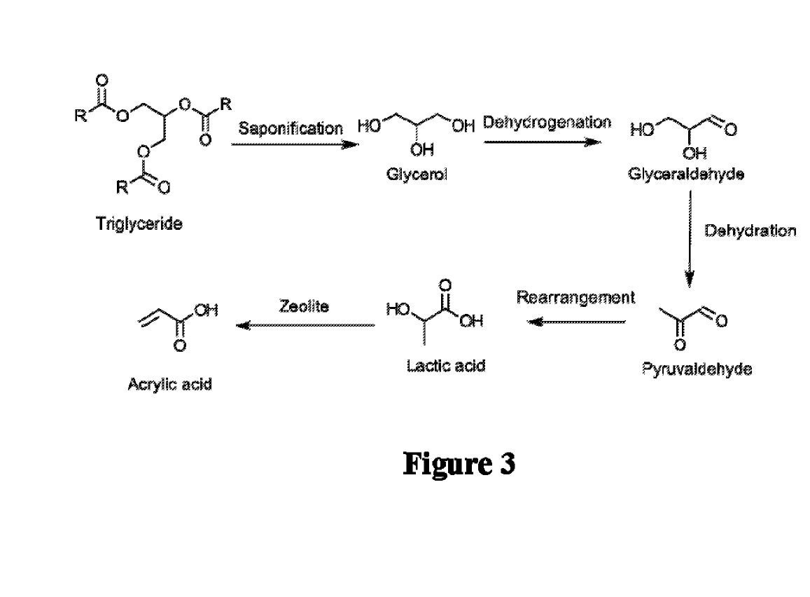

In another example, glycerol may be used as a starting material to produce lactic acid and then acrylic acid using a chemical process without involving any fermentation process (e.g., FIG. 3). Global biodiesel production by trans-esterification of fatty acid esters derived from vegetable oils has increased several fold in the past decade to partly substitute the use of fossil-derived diesel fuel. Glycerol, a byproduct from biodiesel industry, may be a suitable or, in some embodiments, a preferred starting material for the manufacture of acrylic acid and acrylic acid esters according to the processes described in the present invention.

For example, one approach to produce lactic acid from glycerol may use the thermochemical conversion process where at temperatures higher than about 550° C. glycerol converts to lactic acid through intermediary compounds like glyceraldehydes, 2-hydroxypropenal and pyruvaldehyde. However, the thermochemical conversion process can cause significant decomposition of pyruvaldehyde and lactic acid at this elevated temperature, thereby leading to a decrease in the selectivity for lactic acid production. In some instances, the use of a chemical catalyst that mediates the dehydrogenation reaction responsible for the production of lactic acid may allow for the reduction in temperature, thereby enhancing selectivity and mitigating decomposition. In some instances, a heterogeneous catalyst may be preferred as the heterogeneous catalyst may be recovered and reused multiple times, may not require any buffering, and may be easily modified to run on a continuous flow process mode instead of a batch process mode to increase throughput and turnover time. These advantages may translate to a significant reduction in operating costs and waste disposal.

The heterogeneous catalysts suitable for the conversion of glycerol to lactic acid may comprise metals, which may include, but are not limited to, nickel, cobalt, copper platinum, palladium, ruthenium, rhodium, and any combination thereof. In some embodiments, the heterogeneous catalyst may optionally be supported on a support, which may include, but is not limited to, carbon, silica, alumina, titania, zirconia, zeolites, and the like. In some embodiments, the reaction mixture may further comprise additional hydrogen or oxygen. In some embodiments, the selected catalyst may be utilized without additional hydrogen or oxygen.

In some embodiments, the heterogeneous catalysts comprising metals may be used in the presence of alkaline components, which may include, but are not limited to, an alkali, alkaline earth metal hydroxide, a solid base, and any combination thereof. In some preferred embodiments, the conversion of glycerol to lactic acid may utilize a copper based catalyst with a base promoter but without a reductant or an oxidant in a single pot reaction.

In some embodiments, acrylic acid may be produced from sucrose. In some instances, sucrose may be hydrolyzed to yield glucose and fructose. In some instances it has been observed that fructose may then be directly converted to lactic acid using chemical catalysts with an almost stoichiometric yield while glucose to lactic acid conversion provides a yield of about 64%. As such, some embodiments may involve hydrolyzing sucrose to yield glucose and fructose; isomerizing the glucose to yield fructose; and converting the combined fructose to lactic acid using chemical catalysis. Similarly, some embodiments may involve hydrolyzing starch to yield glucose; isomerizing the glucose to yield fructose; and converting the combined fructose to lactic acid using chemical catalysis.

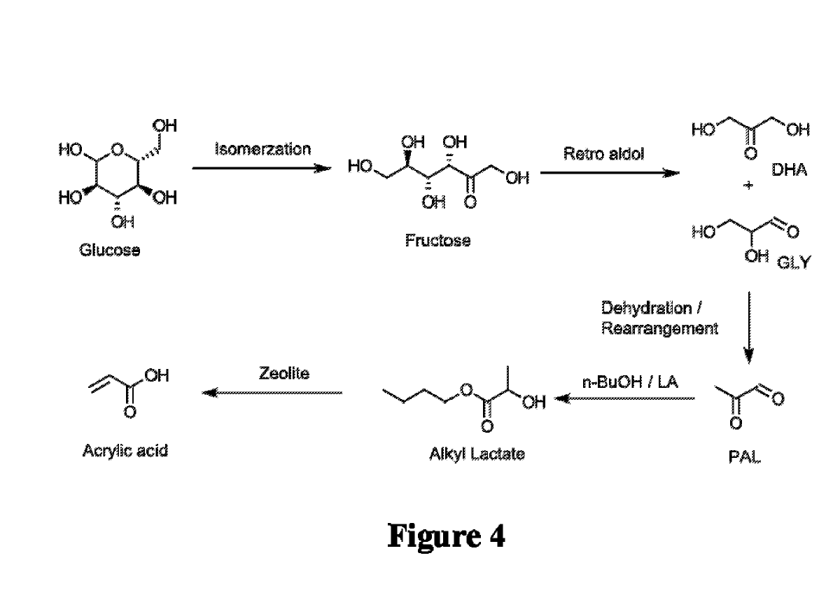

Converting the fructose (from combined or individual sources) to lactic acid using chemical catalysis may involve, as illustrated in FIG. 4, a retro-aldol reaction of fructose to yield dihydroxyacetone (DHA) and glyceraldehydes (GLY), which are together then converted by dehydration and rearrangement into pyruvic aldehyde (PAL). The PAL may further be converted under action of a Lewis acid into the desired alkyl lactate or lactic acid in alcoholic solvents or water. Then, in some embodiments, the lactic acid and alkyl lactate may be converted to acrylic acid and acrylate ester using a zeolite catalyst at about 330° C.

Lewis acidic zeolites, e.g., Sn-Beta, have shown surprisingly high activity and selectivity for the conversion of sucrose, glucose, and fructose to esters of lactic acid. In some embodiments, a solid Lewis acidic catalyst may comprise a zeotype material, which in some preferred embodiments further comprises a tetravalent metal, e.g., Sn, Pb, Ge, Ti, Zr, and/or Hf, incorporated in the framework of the zeotype material. For example, a solid Lewis acidic catalyst may comprise a zeotype material and tetravalent Sn and/or tetravalent Ti.

Example of suitable zeotype materials may include, but are not limited to, a structure type BEA, MFI, MEL, MTW, MOR, LTL, or FAU, such as zeolite beta and ZSM-5. Another example of suitable zeotype materials may include TS-1. These various examples of zeotype materials may be Lewis acidic mesoporous amorphous materials, which, in some embodiments, may preferably have the structure type of MCM-41 or SBA-15.

The reactions for the production of lactic acid from sucrose, glucose, and fructose may be conducted in a batch mode or in a flow reactor at temperatures ranging from about 50° C. to about 300° C., preferably about 100° C. to about 220° C., and most preferably about 140° C. to about 200° C.

Referring again to the reaction pathways of the present invention, in some embodiments, starting compositions described herein may comprise reactants and solvents. Solvents suitable for use in conjunction with reactants described herein may include, but are not limited to, water, alcohols (e.g., methanol, ethanol, propanol, iso-propanol, n-propanol, butanol, iso-butanol, n-butanol, 2-ethylhexanol, iso-nonanol, iso-decylalcohol, or 3-propylheptanol), tetrahydrofuran, methylene chloride, toluene, xylene, and the like, and any combination thereof. In some embodiments, the solvent may be in a supercritical state.

In some embodiments, starting compositions described herein may comprise reactants in a concentration ranging from a lower limit of about 5%, 10%, 15%, 25%, or 50% by weight of the starting composition to an upper limit of about 95%, 90%, 85%, 75%, or 50% by weight of the starting composition, and wherein the concentration may range from any lower limit to any upper limit and encompass any subset therebetween. In some embodiments, starting compositions described herein may comprise solvents in a concentration ranging from a lower limit of about 5%, 10%, 15%, 25%, or 50% by weight of the starting composition to an upper limit of about 95%, 90%, 85%, 75%, or 50% by weight of the starting composition, and wherein the concentration may range from any lower limit to any upper limit and encompass any subset therebetween.

In some embodiments, when the reactant comprises an ester (e.g., an ester of lactic acid), starting compositions described herein may have a water content ranging from a lower limit of about 1%, 2%, 3%, or 4% by weight of the starting composition to an upper limit of about 10%, 9%, 8%, 7%, 6%, or 5% by weight of the starting composition, and wherein the water content may range from any lower limit to any upper limit and encompass any subset therebetween. By way of nonlimiting example, a starting composition may comprise methyl lactate, methanol, and water, wherein the water content is about 3.5% to about 5% by weight of the starting composition. By way of nonlimiting example, a starting composition may comprise butyl lactate, butanol, and water, wherein the water content is about 1% to about 5% by weight of the starting composition.

In some embodiments, starting compositions described herein may be subjected to one or more additional process steps prior to beginning a reaction pathway of the present invention. Examples of additional process steps may include, but are not limited to, filtration, acidification, crystallization, pervaporation, electrodialysis, ion exchange, liquid-liquid extraction, and simulated moving bed chromatography. By way of nonlimiting example, additional process steps may be utilized to enrich the lactic acid content and to remove the impurities from the fermentation broth in which the lactic acid was produced.

Some of the reactions described herein (e.g., dehydroxylation reactions and/or esterification reactions) involve contacting reactants and/or intermediates with catalysts. In some embodiments, contacting may occur inside a system (e.g., introducing starting compositions into a reactor that holds catalysts), outside the system (e.g., mixing catalysts to starting compositions prior to introduction into a reactor), and any combination thereof. Systems and processes are described in more detail herein.

In some embodiments, a dehydroxylation reaction useful in reaction pathways of the present invention may involve contacting reactants and/or intermediates with dehydroxylation catalysts described herein. In some embodiments, dehydroxylation catalysts described herein may be a liquid, a solid, or a combination thereof. In some embodiments, the reactants and/or intermediates may be in the vapor phase and/or in the liquid phase. By way of nonlimiting example, in some embodiments, a dehydroxylation reaction useful in reaction pathways of the present invention may involve contacting reactants and/or intermediates in the vapor phase with solid dehydroxylation catalysts.

Dehydroxylation catalysts suitable for use in conjunction with the present invention may, in some embodiments, include, but are not limited to, zeolites, modified zeolites, acid catalysts, weak acid catalysts, strong acid catalysts, neutral catalysts, basic catalysts, ion exchange catalysts, zeolites, solid oxides, and the like, and any combination thereof. In some embodiments, preferred dehydroxylation catalysts may include, but are not limited to, L-type zeolites and/or modified zeolites. As used herein, zeolites refer to the aluminosilicate members of the family of microporous solids known as “molecular sieves.” Zeolites have a general molecular formula M_{x/n}[(AlO₂)ₓ(SiO₂)_{y}] z H₂O where n is the charge of the metal cation (M), M is usually Na⁺, K⁺ or Ca²⁺, and z is the number of moles of water of hydration which is highly variable. An example of a zeolite may be natrolite with the formula Na₂Al₂Si₃O₁₀ 2 H₂O. As used herein, the term “modified zeolites” refer to zeolites having been modified by (1) impregnation with inorganic salts and/or oxides and/or (2) ion exchange.

It is believed that zeolites contain channels (also known as voids or pores) that are occupied by the cations and water molecules. Without being limited by theory, it is believed that dehydroxylation reactions conducted in the presence of zeolites may take place preferentially within the channels of the zeolites. Accordingly, it is believed that the dimensions of the channels affect, inter alia, the diffusion rates of chemicals therethrough, and consequently the selectivity and conversion efficiency of the dehydroxylation reactions. In some embodiments, the diameter of the channels in zeolite catalysts suitable for use in conjunction with dehydroxylation reactions disclosed herein may range from about 1 to about 20 angstroms, or more preferably about five to about 10 angstroms, including any subset therebetween.

Zeolites suitable for use as dehydroxylation catalysts described herein may be derived from naturally-occurring materials and/or may be chemically synthesized. Further, zeolites suitable for use as dehydroxylation catalysts described herein may have, in some embodiments, crystalline structures commensurate with L-type zeolites, Y-type zeolites, X-type zeolites, and any combination thereof. Different types of zeolites such as A, X, Y, and L differ from each other in terms of their composition, pore volume, and/or channel structure. A-type and X-type zeolites have a molar ratio of Si to Al of about 1 and a tetrahedral aluminosilicate framework. Y-type zeolites have a molar ratio of Si to Al of about 1.5 to about 3.0 and a framework topology similar to that of X-type zeolites. L-type zeolites have a molar ratio of Si to Al of about 3.0 and have one-dimensional pores of about 0.71 nm aperture leading to cavities of about 0.48 nm×1.24 nm×1.07 nm.

In some embodiments, modified zeolites may be produced by performing an ion exchange with a zeolite. In some embodiments, modified zeolites suitable for use as dehydroxylation catalysts described herein may have ions associated therewith that may include, but are not limited to, H⁺, Li⁺, Na⁺, K⁺, Cs⁺, Mg²⁺, Ca²⁺, La²⁺, La³⁺, Ce²⁺, Ce³⁺, Ce⁴⁺, Sm²⁺, Sm³⁺, Eu²⁺, Eu³⁺, and the like, and any combination thereof. As used herein, “[ions associated]-[crystalline structure]-type zeolite” is used to abbreviate specific zeolites and/or modified zeolites. For example, an L-type zeolite having potassium ions associated therewith is abbreviated by K-L-type zeolite. In another example, a X-type zeolite having potassium and sodium ions incorporated therewith is abbreviated Na/K—X-type zeolite. In some embodiments, L-type zeolites may be modified by techniques like calcination, ion exchange, incipient wetness impregnation, hydro-treatment with steam, any hybrid thereof, and any combination thereof.

In some embodiments, modified zeolites suitable for use as dehydroxylation catalysts described herein may have more than one cation associated therewith. In certain embodiments, modified zeolites suitable for use as dehydroxylation catalysts described herein may comprise a first cation and a second cation, where the mole ratio of the first cation to the second cation may range from a lower limit of about 1:1000, 1:500, 1:100, 1:50, 1:10, 1:5, 1:3, 1:2, or 1:1 to an upper limit of about 1000:1, 500:1, 100:1, 50:1, 10:1, 5:1, 3:1, 2:1, or 1:1, and wherein the mole ratio may range from any lower limit to any upper limit and encompass any subset therebetween. By way of nonlimiting examples, modified zeolites suitable for use as dehydroxylation catalysts described herein may, in some embodiments, be H/Na-L-type zeolites, Li/Na—X-type zeolites, Na/K—Y-type zeolites, and any combination thereof. By way of another nonlimiting example, modified zeolites suitable for use as dehydroxylation catalysts described herein may, in some embodiments, be Na/K-L-type zeolites, Na/K—Y-type zeolites, and/or Na/K—X-type zeolites, where the ratio of sodium ions to potassium ions is about 1:10 or greater.

Without being limited by theory, it is believed that in embodiments where at least some H⁺ ions on the zeolite are exchanged, the catalyst acidity of the produced modified zeolite may be reduced. The magnitude of the reduction in acidity may be determined using a suitable test. For example, ASTM (American Society for Testing and Materials) D4824 may be used to determine the acidity of the modified zeolite. Briefly, this test uses ammonia chemisorption to determine the acidity of the modified zeolite where a volumetric system is used to obtain the amount of chemisorbed ammonia.

In some embodiments, modified zeolites may be zeolites impregnated with an inorganic salt and/or oxide thereof. Inorganic salts suitable for use in producing modified zeolites describe herein may, in some embodiments, include, but are not limited to, phosphates, sulfates, molybdates, tungstates, stanates, antimonates, and the like, and any combination thereof with a cation of calcium, sodium, magnesium, aluminum, potassium, and the like, and any combination thereof. By way of nonlimiting example, in some embodiments, modified zeolites may be produced with sodium phosphate compounds (e.g., monosodium phosphate (NaH₂PO₄), disodium phosphate (Na₂HPO₄), and trisodium phosphate (Na₃PO₄)), potassium phosphate compounds, sodium aluminum phosphate compounds (e.g., Na₈Al₂(OH)₂(PO₄)₄), and any combination thereof.

In some embodiments, modified zeolites suitable for use as dehydroxylation catalysts described herein may be impregnated with an inorganic salt and/or oxide thereof at a concentration ranging from a lower limit of about 0.1 mmol, 0.2 mmol, or 0.4 mmol per gram of modified zeolite to an upper limit of about 1.0 mmol, 0.8 mmol, or 0.6 mmol per gram of modified zeolite, and wherein the concentration may range from any lower limit to any upper limit and encompass any subset therebetween. By way of nonlimiting example, impregnated L-type zeolites suitable for use in conjunction with dehydroxylation reactions described herein may be Na/K-L-type zeolite impregnated with a sodium phosphate compound, where the ratio of sodium ions to potassium ions is about 1:10 or greater.

One of ordinary skill in the art should recognize additional steps for the preparation of modified zeolites by ion exchange and/or impregnation. For example, drying and/or calcining may be needed to, inter alia, remove water from the pores and/or convert salts to oxides thereof. Further, proper storage may be needed to, inter alia, prevent the modified zeolites from being at least partially deactivated during storage. As used herein, the term “calcining” refers to a process by which the zeolite catalyst is subjected to a thermal treatment process in the presence of air for the removal of a volatile fraction.

Acid catalysts suitable for use as dehydroxylation catalysts described herein may be liquids, solids, or a combination thereof. Examples of liquid acid catalysts suitable for use as dehydroxylation catalysts described herein may include, but are not limited to, sulfuric acid, hydrogen fluoride, phosphoric acid, paratoluene sulfonic acid, and the like, and any combination thereof.

In some embodiments, solid acid catalysts may be obtained by contacting a hydroxide or hydrated hydroxide of a metal belonging to group IV of the Periodic Table with a solution containing a sulfurous component and calcining the mixture between about 350° C. to about 800° C. The solid acid catalysts may, in some embodiments, have acidity higher than that of 100% sulfuric acid. In some embodiments, solid acid catalysts may be preferred over liquid acid catalysts because, inter alia, solid acid catalysts may exhibit higher catalyzing power and lower corrosiveness while advantageously being easier to remove when the reaction is completed.

Weak acid catalyst suitable for use as dehydroxylation catalysts described herein may include, but are not limited to, titania catalysts, SiO₂/H₃PO₄ catalysts, fluorinated Al₂O₃ (e.g., Al₂O₃.HF catalysts), Nb₂O₃/SO₄⁻² catalysts, Nb₂O₅.H₂O catalysts, phosphotungstic acid catalysts, phosphomolybdic catalyst, silicomolybdic acid catalysts, silicotungstic acid catalysts, acidic polyvinylpyridine hydrochloride catalysts (e.g., PVPH⁺Cl⁻® available from Reilly), hydrated acidic silica catalysts (e.g., ECS-3® available from Engelhard), and the like, and any combination thereof.

Basic catalysts suitable for use as dehydroxylation catalysts described herein may include, but are not limited to, ammonia, polyvinylpyridine, metal hydroxide, Zr(OH)₄, amines with the general formula NR¹R²R³ (where R¹, R², and R³ are independently selected from the group of side chain or functional groups including, but not limited to, hydrogen, hydrocarbons containing from 1 to 20 carbon atoms, alkyl, and/or aryl groups containing from 1 to 20 carbon atoms), and the like, and any combination thereof. In some embodiments, ammonium lactate may advantageously be used as a reactant for acrylic acid production because when subjected to high temperature treatment ammonium lactate decomposes to release ammonia (a basic catalyst) and lactic acid.

Solid oxides suitable for use as dehydroxylation catalysts described herein may include, but are not limited to, TiO₂ (e.g., Ti-0720® available from Engelhard), ZrO₂, Al₂O₃, SiO₂, ZnO₂, SnO₂, WO₃, MnO₂, Fe₂O₃, V₂O₅, SiO₂/Al₂O₃, ZrO₂/WO₃, ZrO₂/Fe₂O₃, ZrO₂/MnO₂, and the like, and any combination thereof. It should be noted that the description above relating to ion exchange and impregnation in relation to L-type zeolites applies in its entirety to solid oxides.

Solid dehydroxylation catalysts suitable for use as dehydroxylation catalysts described herein may, in some embodiments, have a high surface area. In some embodiments, solid dehydroxylation catalysts suitable for use as dehydroxylation catalysts described herein may have a surface area of about 100 m²/g or greater. In some embodiments, solid dehydroxylation catalysts suitable for use as dehydroxylation catalysts described herein may have a surface area ranging from a lower limit of about 100 m²/g, 125 m²/g, 150 m²/g, or 200 m²/g to an upper limit of about 500 m²/g, 400 m²/g, 300 m²/g, or 250 m²/g, and wherein the surface area may range from any lower limit to any upper limit and encompass any subset therebetween.

In some embodiments, dehydroxylation catalysts described herein may be present in dehydroxylation reactions described herein in a molar ratio of catalyst to reactant/intermediate of about 1:1000 or greater. In some embodiments, dehydroxylation catalysts described herein may be present in dehydroxylation reactions described herein in a molar ratio of catalyst to reactant/intermediate ranging from a lower limit of about 1:1000, 1:500, or 1:250 to an upper limit of about 1:1, 1:10, or 1:100, and wherein the molar ratio may range from any lower limit to any upper limit and encompass any subset therebetween.

In some embodiments, dehydroxylation reaction may utilize more than one type of dehydroxylation catalyst described herein. In some embodiments, the weight ratio of the two dehydroxylation catalysts may be about 1:10 or greater. In some embodiments, the weight ratio of the two dehydroxylation catalysts may range from a lower limit of about 1:10, 1:5, 1:3, or 1:1 to an upper limit of about 10:1, 5:1, 3:1, or 1:1, and wherein the weight ratio may range from any lower limit to any upper limit and encompass any subset therebetween. One skilled in the art with the benefit of this disclosure should understand the extension of such ratios to three or more dehydroxylation catalysts described herein.

In some embodiments, a dehydroxylation reaction useful in reaction pathways of the present invention may be performed at a temperature ranging from a lower limit of about 100° C., 150° C., or 200° C. to an upper limit of about 500° C., 400° C., or 350° C., and wherein the temperature may range from any lower limit to any upper limit and encompass any subset therebetween.

Polymerization inhibitors may be utilized in conjunction with dehydroxylation reactions described herein to prevent the polymerization of α,β-unsaturated carboxylic acids or the esters thereof produced along the reaction pathway. In some embodiments, polymerization inhibitors may be introduced to a reaction pathway of the present invention, e.g., in the starting composition, during a dehydroxylation reaction, during an esterification reaction, and any combination thereof. Examples of polymerization inhibitors may include, but are not limited to, 4-methoxy phenol, 2,6-di-tert-butyl-4-methylphenol, sterically hindered phenols, and the like.

In some instances, the dehydroxylation reaction of the present invention can be conducted in the absence of any dehydroxylation catalyst described herein and only in the presence of inert solid support such as glass, ceramic, porcelain, or metallic material present within the reaction vessel. In some embodiments, supercritical solvents may be useful in starting compositions for dehydroxylation reactions conducted in the absence of a dehydroxylation catalyst described herein.

In some embodiments, an esterification reaction useful in reaction pathways of the present invention may involve contacting reactants and/or intermediates with alcohols (as reactants and/or solvents) and esterification catalysts described herein.

In some embodiments, an esterification reaction may be performed in the presence of an alcohol, as a solvent and/or as a reactant. In some embodiments, examples of suitable alcohols may include, but are not limited to, alkyl alcohols (e.g., C₁-C₂₀ alcohols) (e.g., methanol, ethanol, propanol, iso-propanol, n-propanol, butanol, iso-butanol, n-butanol, 2-ethylhexanol, iso-nonanol, iso-decylalcohol, or 3-propylheptanol), aryl alcohols (e.g., benzyl alcohol, and the like), cyclic alcohols (e.g., cyclohexanol, cyclopentanol, and the like), and any combination thereof.

In some embodiments, a fermentation broth described herein containing a salt of α-hydroxy carboxylic acid described herein (e.g., ammonium lactate) may be used as the reactant for an esterification reaction. Use of such a salt may necessitate a two-step esterification reaction involving, for example, decomposing the ammonium lactate into ammonia and lactic acid and then reacting the lactic acid with an alcohol as described herein. Because both steps of this esterification reaction are reversible and may reach an equilibrium, in some embodiments, excess reactants may be utilized and/or products may be continuously removed so as to minimize the reverse reaction and enhance overall yield.

In some embodiments, esterification reactions described herein may be carried out in, inter alia, batch processes or reactive distillation processes. In esterification reactions described herein, including by the aforementioned processes, to improve the efficiency of the esterification process, an esterification catalyst may be used. In some embodiments, the catalyst may be a homogeneous catalyst or a heterogeneous catalyst. Homogenous catalysts suitable for improving the rate of the esterification process of the present invention may, in some embodiments, include, but are not limited to, strong mineral acids, strong organic acids, and any combination thereof. In some preferred embodiments, the homogeneous catalyst for use in conjunction with esterification reactions described herein may be hydrochloric acid. Heterogeneous catalysts suitable for improving the rate of the esterification process of the present invention may, in some embodiments, include, but are not limited to, cationic resin catalysts, for example, AMBERLYST-15 catalyst (a strongly acidic, sulfonic acid, macroeticular polymeric resin based on crosslinked styrene divinylbenzene copolymers, available from Rohm and Haas). In some embodiments, an esterification catalyst described herein may comprise a mixture of tin salt and aluminate, wherein the tin salt is capable of reacting with the aluminum salt to form stannous aluminate and/or provide a stannous ion. In some embodiments, an esterification catalyst described herein may comprise a mixture of stannous salt, aluminate, and finely divided sand. In some embodiments, an esterification catalyst described herein may comprise gaseous CO₂.

The rate of esterification reaction may be changed by a number of different ways. For example, an increase in the concentration of the catalyst, an increase in the ratio of alcohol to lactic acid, and/or increased temperatures may be used to increase the rate of esterification. One skilled in the art with the benefit of this disclosure should understand the considerations when changing the rate of an esterification reaction, e.g., increasing the temperature may cause volatile reactants like some alcohols to evaporate, which may be remedied with, for example, a condenser.

In some embodiments, the pressure of an esterification reaction described herein may range from about 1 atmosphere to about 10 atmospheres. For example, such a pressure should be sufficient to maintain the ammonium lactate and alcohol in the reaction mixture in the liquid phase.

In some embodiments that utilize ammonium lactate reactants, the molar ratio of alcohol to ammonium lactate in the esterification reaction may be from about 1:1 to about 10:1, or more preferably about 1:1 to about 5:1, and encompasses any subset therebetween.

In some embodiments, the efficiency of esterification reaction may be effected by the amount of water present. In some embodiments, a drying reagent may be utilized in an esterification reaction described herein so as to reduce the water content. In some embodiments, the drying reagent may extract water from the vapor phase during the recovery of alcohol. In some embodiments, the drying reagent may function by the adsorption and absorption of water into the drying agent. Preferred drying reagents do not absorb alcohols, especially those being utilized in the esterification reaction. In some embodiments, the drying reagent may be an inert porous substance. Exemplary drying reagents may include, but are not limited to, diatomaceous earth, molecular sieve, zeolites, and the like, and any combination thereof. Commercially available substances having a surface area of between approximately 12 cm²/gram to 20 cm²/gram may, in some embodiments, also be appropriate for use as drying agents.

Controlling water content during an esterification reaction may also be achieved and/or enhanced by using hydrophilic pervaporation membranes. The use of pervaporation membranes during the esterification reaction may advantageously provide multiple functions including, but not limited to, assisting in the control of the water content, minimizing the reverse reaction, and assisting in the separation of the lactic acid ester by distillation.

Controlling water content during an esterification reaction may also be achieved and/or enhanced by using azeotroping agent, e.g., benzene.

In some embodiments, any combination of the aforementioned may be used in controlling the water content during an esterification reaction described herein.

In some embodiments that utilize ammonium lactate reactants, ammonia may be released during the esterification reaction. In some embodiments, the esterification reaction may be carried out at high temperature with reflux, and, an inert gas may be used to remove the ammonia from the condenser. In some embodiments, the ammonia released during the downstream processing of the fermentation broth containing ammonium lactate can be condensed, compressed and recycled into a fermentation vessel as a source of alkali to maintain the pH of the microbial growth medium.

At larger scale, reactive distillation may, in some embodiments, be used with a resin esterification catalyst to minimize the reverse reaction of the esterification reaction as described above, and thereby producing high purity esters with purity closer to 100% theoretical yields. In some embodiments, the lactic acid may be dissolved (or dispersed) in an alcohol and then passed through a reaction zone with a packed bed of an esterification catalyst. The product stream from the outlet of the reaction zone, which contains the excess alcohol and the desired final product, may then be fed to a distillation column for the separation and purification of the ester formed. The lactic acid ester with higher boiling point may, in some embodiments, be separated from the alcohol through fractional distillation.

In some embodiments, esterification catalysts described herein may be a liquid, a solid, or a combination thereof. In some embodiments, the reactants and/or intermediates may be in the vapor phase and/or in the liquid phase. In some embodiments, esterification catalysts suitable for use in conjunction with reaction pathways of the present invention may include, but are not limited to, ion exchange resins, aluminum silicate compounds (e.g., zeolites and/or modified zeolites described herein), and the like, and any combination thereof.

Under certain circumstances, an esterification reaction may be carried out in the absence of any exogenous catalysts. The esterification reaction in the absence of any exogenous catalyst is preferred. As used herein, the term “exogenous catalyst” refers to the chemical entity which is added to any chemical reaction from an outside source in order to lower the activation energy required for chemical reaction and to improve the overall rate of the chemical reaction. This term “exogenous catalyst” is used to distinguish the situation wherein some of the substrates of the chemical reaction itself can act as a catalyst. The esterification reaction may, in some embodiments, be carried out without the addition of any exogenous catalyst as explained in detail in International Patent Application No. PCT/US11/50707 entitled “Catalytic Dehydration of Lactic Acid and Lactic Acid Esters,” the entirety of which is incorporated herein by reference. For example, in some embodiments, the fermentation broth may be heated to about 100° C. in the presence of appropriate alcohol without the addition of any exogenous catalyst to achieve the formation of lactic acid ester.

Examples of ion exchange resins suitable for use in conjunction with esterification reactions described herein may, in some embodiments, include, but are not limited to, an AMBERLYST® product, e.g., AMBERLYST® 70 (a strong acid ion exchange resin, available from Rohm and Haas and Dow Chemicals).

In some embodiments, esterification reactants and dehydroxylation reactants may be used in the same reaction vessel to allow for concurrent reaction pathways of the present invention (e.g., Reaction Pathway 4 of FIG. 1).

In some embodiments, esterification reactants and dehydroxylation reactants may be used in a sequence of two different reaction vessels to allow for sequential reaction pathways of the present invention (e.g., Reaction Pathway 1 or 2 of FIG. 1). Alternately, the dehydroxylation reaction and the esterification reactions can be carried out in the same reactor in sequence. For example, Reaction Pathway 1 may be carried out such that the top portion of a reactor chamber contains the esterification catalyst, the bottom portion of the reactor chamber contains the dehydroxylation catalyst, and the two catalysts are separated by an inert material in the middle. The reactant along with appropriate alcohol may, in some embodiments, be introduced on the top of the reactor chamber. As the reactant passes through the esterification catalyst, the reactant is esterified and the ester thus formed passes through inert material and reaches the lower portion of the reactor chamber wherein the dehydroxylation reaction occurs leading to the formation of an ester of an α,β-unsaturated carboxylic acid, e.g., acrylic acid ester for a lactic acid reactant.

In some embodiments, the dehydroxylation catalyst may occupy the upper part of the reactor vessel and the esterification catalyst may occupy the lower portion of the reactor vessel with an inert material therebetween, which may be useful in carrying out Reaction Pathway 2. For example, the reactant may be supplied to the top of the reactor, and then the α,β-unsaturated carboxylic acid produced on the upper portion of the reactor vessel may pass through the inert material and enter the lower portion of the reactor and be contacted with the esterification catalyst and an alcohol so as to yield an ester of the α,β-unsaturated carboxylic acid, which may be collected at the bottom of the reactor. In some embodiments, the alcohol may be in the vapor phase. In some embodiments where the dehydroxylation catalyst and the esterification catalyst are the same, both the upper portion and the lower portion of the reactor is filled with the catalyst and the inert material disposed therebetween may be optional.

In some embodiments, the esterification reaction and the dehydroxylation reactions may occur concurrently in the same reaction vessel as described in Reaction Pathway 4. In some embodiments to achieve concurrent reaction, the reactants including the desired alcohol may be introduced simultaneously at the top of a reactor vessel, and the corresponding ester of an α,β-unsaturated carboxylic acid may be collected at the bottom of the reactor vessel.

In some embodiments, an aluminum silicate compound (e.g., a zeolite and/or a modified zeolite) may function both as an esterification and a dehydroxylation catalyst. In some embodiments, the catalyst that functions both as an esterification and a dehydroxylation catalyst may be utilized in sequential reaction pathways of the present invention (e.g., Reaction Pathway 1 and 2 of FIG. 1) or concurrent reaction pathways of the present invention (e.g., Reaction Pathway 4 of FIG. 1).

In some embodiments, an esterification reaction useful in reaction pathways of the present invention may be performed at a temperature ranging from a lower limit of about 50° C., 100° C., 150° C., or 200° C. to an upper limit of about 500° C., 400° C., or 350° C., and wherein the temperature may range from any lower limit to any upper limit and encompass any subset therebetween.

In some embodiments, reaction pathways of the present invention may be utilized to produce α,β-unsaturated carboxylic acids or the esters thereof. Examples of α,β-unsaturated carboxylic acids or the esters thereof may include, but are not limited to, acrylic acid, alkyl esters of acrylic acid (e.g., methyl acrylate and butyl acrylate), and the like, and any combination thereof.

Surprisingly, it has been found that the use of butyl lactate in the starting composition reduces undesired byproduct formation that is seen with the use of other alkyl lactates such as methyl lactate, and that butyl lactate is preferentially converted into acrylic acid rather than butyl acrylate.

In some embodiments, the reaction pathways of the present invention may produce aldehydes (e.g., acetaldehyde). Accordingly, a reaction pathway may optionally further comprises an oxidizing reaction to convert acetaldehyde to acetic acid.

In some embodiments, a reaction pathway of the present invention may have a conversion efficiency of about 40% or greater, in some embodiments about 50% or greater, in some embodiments about 55% or greater, in some embodiments about 60% or greater, in some embodiments about 65% or greater, in some embodiments about 70% or greater, in some embodiments about 75% or greater, in some embodiments about 80% or greater, in some embodiments about 85% or greater, in some embodiments about 90% or greater, in some embodiments about 95% or greater, in some embodiments about 98% or greater, or in some embodiments about 99% greater.

In some embodiments, the selectivity of the reaction pathway of the present invention may result in production of α,β-unsaturated carboxylic acids and/or esters thereof in an amount that is 40 mole % or greater of a product, in some embodiments 50 mole % or greater of a product, in some embodiments 55 mole % or greater of a product, in some embodiments 60 mole % or greater of a product, in some embodiments 65 mole % or greater of a product, in some embodiments 70 mole % or greater of a product, in some embodiments 75 mole % or greater of a product, in some embodiments 80 mole % or greater of a product, in some embodiments 85 mole % or greater of a product, in some embodiments 90 mole % or greater of a product, in some embodiments 95 mole % or greater of a product, in some embodiments 98 mole % or greater of a product, and in some embodiments 99 mole % or greater of a product.

It should be understood that the conversion efficiency and/or selectivity of the reaction pathway is dependent on, inter alia, controlling the temperature for calcining the catalyst where applicable, the composition of the dehydroxylation and/or esterification catalysts, the concentration of reactants and/or intermediates, and/or the duration of the contact between the reactants and/or intermediates and the dehydroxylation and/or esterification catalysts.

In some instances, it has been observed that the reactor metallurgy may adversely affect the acrylic acid selectivity in lactic acid dehydroxylation reaction. Without being limited by theory, it is believed that the lactic acid feed may cause the corrosion of reactor walls leading to the leaching of metal components from the reactor wall. For example, when a stainless steel reactor is used in the dehydroxlation reaction, metal components such as nickel, chromium and iron may leach out into the product stream and/or accumulate onto the dehydroxylation catalyst, which can, for example, be detected using inductively coupled plasma (ICP) analysis. The leached metals may act as a catalyst capable of forming byproducts. For example, nickel released from the walls of a stainless steel reactor may act as a hydrogenation catalyst leading to the formation of propionic acid from acrylic acid. Similarly, iron released from the walls of the stainless steel reactor may function as a decarboxylation catalyst leading to the formation of acetaldehyde. Additionally, some of the components leached out of the reactor walls may lead to the polymerization of lactic acid and acrylic acid. Accordingly, in some embodiments, reactor materials may be chosen to be resistant to corrosion either by feed or the products formed through catalytic dehydroxylation reaction. Examples of suitable reactor materials that may mitigate unwanted catalysis may include, but are not limited to, titanium, silanized stainless steel, quartz, and the like. Such a reactor with reduced level of corrosion may provide for higher selectivity for acrylic acid and reducing byproduct formation.

By way of nonlimiting example, similar to Reaction Pathways 2 and 3 illustrated in FIG. 1, some embodiments may involve first performing a dehydroxylation reaction by contacting a starting composition as described herein with a dehydroxylation catalyst that comprises an L-type zeolite, thereby producing an α,β-unsaturated carboxylic acid and/or ester thereof. Where an α,β-unsaturated carboxylic acid is produced, some embodiments may further involve performing an esterification reaction by contacting the α,β-unsaturated carboxylic acid with an esterification catalyst and an alcohol, thereby producing an α,β-unsaturated carboxylic acid ester. Specifically, in some embodiments, the starting composition may comprise lactic acid, the product of the dehydroxylation reaction may then comprise acrylic acid, and the product of the esterification reaction, should it be performed, may then comprise an acrylic acid ester.

By way of another nonlimiting example, similar to Reaction Pathway 1 illustrated in FIG. 1, some embodiments may involve first performing an esterification reaction by contacting a starting composition that comprises a carboxylic acid derivative as described herein with an esterification catalyst and an alcohol, thereby producing an ester derivative; and second performing a dehydroxylation reaction by contacting the ester derivative with a dehydroxylation catalyst comprising an L-type zeolite, thereby producing an α,β-unsaturated carboxylic acid ester. Specifically, in some embodiments, the starting composition may comprise lactic acid, the product of the esterification reaction may then comprise a lactic acid ester, and the product of the dehydroxylation reaction may then comprise an acrylic acid ester.

By way of yet another nonlimiting example, similar to Reaction 4 illustrated in FIG. 1, some embodiments may involve concurrently performing dehydroxylation and esterification reactions by contacting a starting composition that comprises a carboxylic acid derivative as described herein with an alcohol and a catalyst that comprises an L-type zeolite, thereby producing an α,β-unsaturated carboxylic acid ester. Specifically, in some embodiments, the starting composition may comprise lactic acid and the product of the concurrent dehydroxylation and esterification reactions may then comprise an acrylic acid ester.

Any suitable systems may be used in conjunction with carrying out the reaction pathways of the present invention. In some embodiments, systems suitable for use in conjunction with carrying out the reaction pathways of the present invention may comprise reactors and optionally comprise at least one of preheaters (e.g., to preheat starting compositions, solvents, reactants, and the like), pumps, heat exchangers, condensers, material handling equipment, and the like, and any combination thereof. Examples of suitable reactors may include, but are not limited to, batch reactors, plug-flow reactors, continuously-stirred tank reactors, packed-bed reactors, slurry reactors, fixed-bed reactors, fluidized-bed reactors, and the like. Reactors may, in some embodiments, be single-staged or multi-staged. Further, reaction pathways of the present invention may be performed, in some embodiments, batch-wise, semi-continuously, continuously, or any hybrid thereof.

As stated above, the reaction pathways or portions thereof may be conducted in the liquid and/or vapor phase. Accordingly, carrier gases (e.g., argon, nitrogen, carbon dioxide, and the like) may be utilized in conjunction with the reaction pathways and/or systems described herein. In some embodiments, the reaction pathways or portions thereof may be conducted in the liquid and/or the vapor phase, which, in some embodiments, may be substantially a single inert gas (e.g., the carrier gas being greater than about 90% of a single carrier gas) or a mixture of multiple inert gases. In some embodiments, the reaction pathways or portions thereof may be conducted in the liquid and/or the vapor phase, which, in some embodiments, may be substantially carbon dioxide (e.g., the carrier gas being greater than about 90% carbon dioxide).

In some embodiments, reaction pathways of the present invention may proceed at a weight hour space velocity (“WHSV”) of about 0.2 hr⁻¹ to about 1.5 hr⁻¹, or more preferably about 0.5 hr⁻¹ to about 1.2 hr⁻¹.

In some embodiments, the product of a reaction pathway of the present invention may comprise α,β-unsaturated carboxylic acids and/or esters thereof and other components (e.g., solvents, polymerization inhibitors, byproducts, unreacted reactants, dehydroxylation catalysts, and/or esterification catalysts). Accordingly, the product of a reaction pathway of the present invention may be separated and/or purified into components of the product (including mixtures of components). In some embodiments, the solvent may be separated from the product of a reaction pathway of the present invention and recycled for reuse. Recycling solvents may advantageously produce less waste and reduce the cost of producing α,β-unsaturated carboxylic acids and/or esters thereof.

Suitable techniques for separation and/or purification may include, but are not limited to, distillation, extraction, reactive extraction, adsorption, absorption, stripping, crystallization, evaporation, sublimation, diffusion separation, adsorptive bubble separation, membrane separation, fluid-particle separation, and the like, and any combination thereof.

One skilled in the art with the benefit of this disclosure should further recognize that at least some of the various dehydroxylation and/or esterification catalysts described herein may be regenerated either in situ or ex situ. For example, in some embodiments, zeolites and/or modified zeolites may be regenerated at elevated temperatures in the presence of oxygen (e.g., air or oxygen diluted in an inert gas).

To facilitate a better understanding of the present invention, the following examples of preferred or representative embodiments are given. In no way should the following examples be read to limit, or to define, the scope of the invention.

### EXAMPLES

### I. Abbreviations and Calculations

Table 1 provides formulas for several calculations used throughout the examples section.

TABLE1

| | |
|---|---|
| Liquid Hourly Space Velocity (“LHSV”) | $LHSV ( mL / {mL}_{C} / h ) = \frac{{F}_{lf}}{{V}_{C}}$ |
| Gas Hourly Gas Space Velocity (“GHSV”) | $GHSV ( mL / {mL}_{C} / h ) = \frac{{F}_{gf}}{{V}_{C}}$ |
| Weight Hourly Space Velocity (“WHSV”) | $WHSV ( {g}_{X} / {g}_{C} / h ) = \frac{{G}_{X}}{{G}_{C} * time}$ |
| Reactant Conversion (“Cnv_{Y}”) | ${Cnv}_{X} ( \% ) = \frac{{[ X ]}_{in} - {[ X ]}_{out}}{{[ X ]}_{in}} \times 100$ |
| Product Selectivity (“Sel_{Y}”) | ${Sel}_{Y} ( \% ) = \frac{{[ Y ]}_{out}}{{[ X ]}_{i n} - {[ X ]}_{out}}$ |

where:
C denotes a catalyst;
mL_{c} denotes volume of catalyst;
mL denotes volume of liquid;
X denotes a reactant;
Y denotes a component of the product;
F_{lf} is the liquid flow rate in mL/h;
F_{gf} is the gas flow rate in mL/h;
V_{C} is the volume of C in the reactor;
G_{X} is the mass of X;
G_{C} is the mass of C in the reactor;
{X} in is the molar concentrations of X in the starting composition;
{X} out is the molar concentrations of X in the exit flow; and
{Y} out is the molar concentration of Y in the exit flow.

### II. Catalysts

A plurality of catalysts were prepared by the following methods. The catalysts used in the examples presented herein were obtained from either from W.R. Grace Company, USA or TOSOH USA, Inc. and subjected to appropriate chemical modifications as needed.

A “H—Y-type zeolite” was prepared as 1/16″ pellets having a SiO₂:Al₂O₃ ratio of 6:1 and Na₂O content of 0.28 wt %. The H—Y-type zeolite was calcined to 500° C. for 3 h and kept in sealed vials in a desiccator until use.

A “Na—Y-type zeolite” was prepared as 1/16″ extrudates having SiO₂:Al₂O₃ ratio of 5:1 and Na₂O content of 13 wt %. The Na—Y-type zeolite was calcined and stored as described for the H—Y-type zeolite.

A “K⁴/Na—Y-type zeolite” was prepared by quadrupled exchange of Na—Y-type zeolite with aqueous KCl solution. Thirty grams of Na—Y zeolite (crushed and sieved to 20-60 mesh particle size) were added slowly to 150 mL of 2 M KCl solution and the suspension was stirred in a Rotavapor at 60° C. for 2 h. The flask was removed, and the supernatant was replaced with fresh KCl solution for the 2^{nd} exchange. The same procedure was repeated two more times. The resulting sample was washed multiple times until free of Cl⁻, dried initially at 30° C. and 60° C. at 2-3 mm Hg for 2 h. The catalyst was then transferred to a vacuum oven and kept at 110° C. overnight, calcined at 500° C. for 3 h, and stored in a desiccator before use.

A “K-L-type zeolite” was prepared to have a SiO₂:Al₂O₃ ratio of 7:9 and 14.4% K₂O dry basis. The K-L-type zeolite was crushed and sieved through 20-60 mesh size. The zeolite material was calcined as described for the H—Y-type zeolite above.

A “Li/K-L-type zeolite” was prepared by a single treatment of 15 g of K-L-type zeolite with 150 mL of 1 M LiCl solution at 30° C. for overnight. The resulting sample was washed multiple times until free of Cl⁻ and then dried initially at 30° C. and at 60° C. at 2-3 mm Hg for 4 h. The catalyst was further dried in a vacuum oven at 110° C. overnight and finally calcined at 450° C. for 3 h.

A “Na/K-L-type zeolite” prepared by slow addition of 15 grams of K-L-type zeolite to 200 mL of 1 M aqueous solution of NaCl. The suspension was stirred at 30° C. for 6 h. After removing the supernatant, the solid was washed multiple times until free of Cl⁻. After drying under vacuum, the catalyst was calcined at 450° C. for 3 h as described for the H—Y-type zeolite above.

A “Na²/K-L-type zeolite” was prepared in the same way as the Na/K-L-type zeolite, but after the first exchange, the zeolite was treated with a second portion of fresh NaCl solution.

A “Na³/K-L-type zeolite” was prepared in the same way as the Na²/K-L-type zeolite, but after the second exchange, the zeolite was treated with a third portion of fresh NaCl solution.

A “Na⁴/K-L-type zeolite” was prepared in the same way as the Na³/K-L-type zeolite, but after the third exchange, the zeolite was treated with a fourth portion of fresh NaCl solution.

A “(7.1% Na₂HPO₄)/K-L-type zeolite” was prepared by an incipient wetness method using the K-L-type zeolite. According to this procedure, a solution of Na₂HPO₄.7H₂O (2.164 g Na₂HPO₄ in 13 mL deionized water) was slowly added to 13 g of the K-L-type zeolite. The resultant slurry was kept in a sealed beaker for 2 h. The impregnated solid was then transferred to a conventional oven and dried at 120° C. overnight.

A “(7.1% Na₂HPO₄)/Na³/K-L-type zeolite” was prepared by the same procedure as the (7.1% Na₂HPO₄)/K-L-type zeolite using the Na³/K-L-type zeolite.

A “(2.13% Na₂HPO₄)/K-L-type zeolite” was prepared by the same procedure as the (7.1% Na₂HPO₄)/K-L-type zeolite using less Na₂HPO₄.

A “(3.55% Na₂HPO₄)/K-L-type zeolite” was prepared by the same procedure as the (7.1% Na₂HPO₄)/K-L-type zeolite using less Na₂HPO₄.

A “(14.0% Na₂HPO₄)/K-L-type zeolite” was prepared by the same procedure as the (7.1% Na₂HPO₄)/K-L-type zeolite using more Na₂HPO₄.

### III. Reaction Protocols

Reaction Protocol I (Continuous Vapor Phase Dehydroxylation of Methyl Lactate).

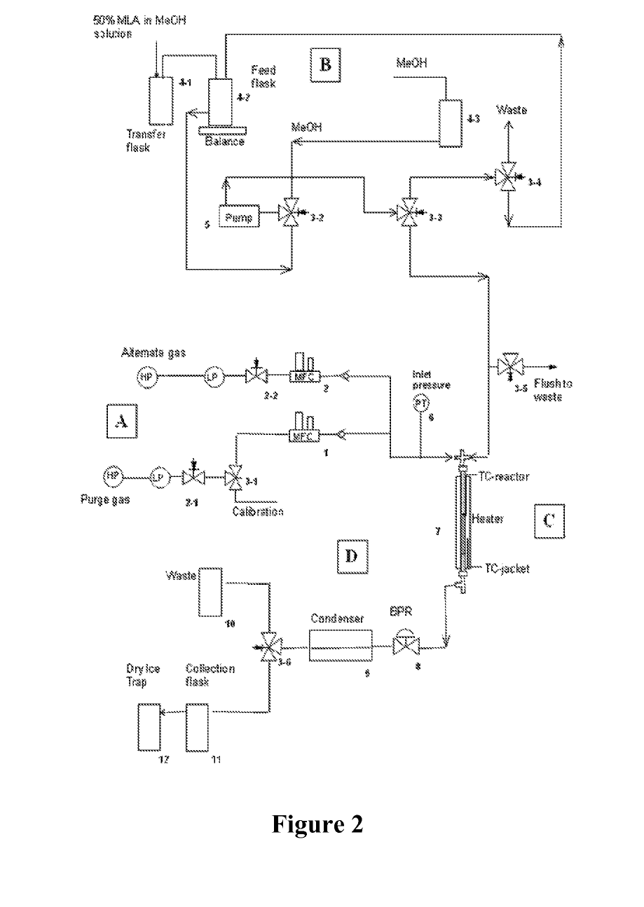

The reaction was carried out in a fixed bed reactor system by passing a starting composition (described specifically in each example below) in the vapor phase over a solid catalyst (described specifically in each example below). A detailed schematic of the reactor system is shown in FIG. 2 and described further herein. The reactor was made of a ½″ by 12″ stainless steel tube, which holds in the bottom section three 10 μm stainless steel filters, serving as support for the catalyst bed. The middle section of the reactor was packed with 10.5 mL of catalyst using a GC column packing vibrator. The top section of the reactor accommodated four of the same inlet filters, thereby providing an 8 mL porous stainless steel contact area for a pre-evaporation and/or gas-liquid mixing. The reactor tube was placed in a column heater (Flatron CH 30) retrofitted with high power heating tape (Omega, 470 W, Part #STH051-060). The temperature of the reactor was monitored by a thermocouple attached near the external wall of the reactor tube and controlled by temperature controller (model M 260, J-KEM Scientific). The liquid hourly velocity (“LHSV”) was varied between about 0.50 h⁻¹ and about 1.10 h⁻¹ (based on the 10.5 mL catalyst volume and about 0.1 to about 0.2 mL/min liquid flow rate). The nitrogen flow rate was varied between 4.4 and 5.6 mL/min.

Reaction Protocol II (Continuous Vapor Phase Dehydroxylation of Butyl Lactate).

The reaction was carried out with a similar procedure and system as described in the methyl lactate dehydroxylation above. The liquid hourly velocity (“LHSV”) was kept constant at 1.2 h⁻¹ (based on the 5 mL catalyst volume and a 0.1 mL/min liquid flow rate). The nitrogen flow rate was also kept constant at 5.0 mL/min.

Reaction Protocol III (Continuous Vapor Phase Dehydroxylation of Lactic Acid).

The reaction was carried out with a similar procedure and system as described in the methyl lactate dehydroxylation above. The reactor was made of a 5/16″ by 6″ stainless steel tube and the catalyst was held between two plugs of Quartz sand (50-70 mesh particle size). In this set of experiments, the liquid hourly space velocity (“LHSV”) was varied in the range about 0.48 h⁻¹ to about 2.4 h⁻¹ (based on 0.04 cc/min liquid flow rate and for catalyst volume in the range of about 1 cc to about 7 cc). The specific reaction parameters are listed for each individual Example further in the text.

Fixed Bed Reactor System.

Referring now to FIG. 2, the system is identified with four separate sections A-D. Section A is a gas control section that includes two individual channels for alternate gas and purge gas. The alternate gas channel provides catalyst pretreatment gas, e.g., ammonia and carbon dioxide, while the purge gas channel provides nitrogen flow in a range of 2-30 mL/min. As shown in Section A, the purge gas channel and the alternate gas channel each have two-way on-off valves 2-1 and 2-2, respectively, and mass flow controllers 1 and 2, respectively. Further, the purge gas channel comprises a three way valve 3-1.

Section B provides for liquid phase handling and consists of two circuits. The first circuit is used for introducing liquid reactants into the system and comprises transfer flask 4-1 and reactant reservoir 4-2. The reactant in transfer flask 4-1 may be transferred to the reactant flask 4-2 by means of positive pressure of inert gas. In this system, all reservoirs are pressurized with nitrogen to 8 psi to allow for smooth operation of pump 5. Additionally, reactant flask 4-2 was placed over a balance for continuous monitoring of the reactant added over time. The second circuit is used for introducing liquid solvent into the system and comprises solvent flask 4-3. The reactants and/or solvents are transferred to pump 5 through a three-way selector valve 3-2. The liquid reactants and/or solvents are then directed by three-way valves, 3-3 and 3-4, to either the reactor 7 or the reactant reservoir 4-2, respectively. This set up allows for the solvent to be used to purge the various transportation lines or to deliver reactant to the reactor 7.

Section C is the reactor 7 as described above in a column heater with a controlled temperature. A second thermocouple may be attached to the reactor for precise monitoring of the reactor temperature.

Section D consists of a spring-loaded back pressure regulator 8, an in-line condenser 9, and a collection flask 11 (a jacketed glass flask in this system). In order to efficiently retain all products having a low boiling point, the temperature of the collection flask 11 was maintained at 4° C. The dry ice trap 12 was placed after the collection flask 11 to quench all products with low boiling points.

### IV. Experiments

Dehydroxylation of Lactic Acid in a Water Solvent.

A starting composition of 40% lactic acid/60% water was reacted in the vapor phase with a variety of catalysts listed in Table 2 at 320° C. for 4 hours (except K-L-type zeolite, which was reacted at 320° C. for 2 hours), according to Reaction Protocol III above. It should be noted that in this section, a reaction time refers to the amount of time that a starting composition is passed over and/or through a bed of catalyst particles, as opposed to, a static sample of starting material and catalyst particles. Further, the conversion and selectivity measurements are based on a sample of the product collected at the reaction time, as opposed to, the total product collected over the entire reaction time.

As shown in Table 2, the Y-type zeolites show high lactic acid conversion at very low acrylic acid selectivity, less than 13%. In contrast, both the K-L-type zeolite and Na³/K-L-type zeolite show good lactic acid conversion of about 65% and high acrylic acid selectivity of about 50%. The Li/K-L-type zeolite increased the lactic acid conversion, but produced significantly more acetaldehyde. The impregnated zeolite, 7.1% Na₂HPO₄/K-L-type zeolite, on the other hand, increased the lactic acid conversion and decreased the acetaldehyde product as compared to the parent K-L-type zeolite.

TABLE 2

| | | Selectivity (mole %) | | |
|---|---|---|---|---|
| | Lactic Acid | Acetal- | Propionic | Acrylic |
| Catalyst | Conversion % | dehyde | Acid | Acid |
| H—Y-type zeolite | 98.3 | 59.7 | NA | 0.00 |
| Na—Y-type zeolite | 73.6 | 43.7 | NA | 19.5 |
| K/Na—Y-type zeolite | 98.5 | 11.1 | NA | 7.8 |
| K-L-type zeolite | 69.3 | 22.3 | NA | 35.9 |
| Na³/K-L-type zeolite | 70.6 | 27.1 | NA | 36.3 |
| Li/K-L-type zeolite | 86.8 | 51.3 | 3.1 | 28.7 |
| 7.1% Na₂HPO₄/K-L-type | 71.7 | 11.7 | 4.2 | 39.9 |
| zeolite | | | | |

Dehydroxylation of Lactic Acid in a Water Solvent.

A starting composition of 40% D,L-lactic acid/60% water with 100 ppm of polymerization inhibitor 4-methoxy phenol was reacted in the vapor phase with a variety of catalysts listed in Table 3 at 320° C. for 4 hours, according to Reaction Protocol III above. As shown in the results shown in Table 3, among the catalysts tested in this experiment, HSZ-500™ KOD1S, an L-type zeolite catalyst, showed a reduced level of conversion but had a higher selectivity for acrylic acid.

TABLE 3

| | Lactic Acid | Selectivity (mole %) | |
|---|---|---|---|
| Catalyst* | Conversion % | Acetaldehyde | Acrylic Acid |
| HSZ-920 ™ HOD1A | 99.6 | 59.5 | 0.2 |
| (a beta zeolite) | | | |
| HSZ-930 ™ HOD1S | 99.9 | 73.8 | 0.5 |
| (a beta zeolite) | | | |
| HSZ-500 ™ KOD1S | 74.7 | 21.8 | 35.4 |
| (L-type zeolite) | | | |
| HSZ-640 ™ HOD1A | 98.2 | 68.2 | 1.6 |
| (a mordenite zeolite) | | | |
| HSZ-330 ™ HUD1A | 98.3 | 59.7 | 0.0 |
| (Y-type zeolite) | | | |
| HSZ-360 ™ HUD1C | 99.2 | 71.4 | 1.2 |
| (Y-type zeolite) | | | |

*Each of these zeolite catalysts were purchased from TOSOH USA, Inc. The catalysts were utilized without any modification.

Dehydroxylation of Lactic Acid in a Water Solvent.

A starting composition of 40% D,L-lactic acid/60% water with 100 ppm of polymerization inhibitor 4-methoxy phenol was reacted in the vapor phase with a variety of catalysts listed in Table 4 at 320° C. for 4 hours, according to Reaction Protocol III above.

As shown in Table 4, modified zeolites with smaller cations may have higher conversion rates, but the selectivity towards acrylic acid appears to be negatively impacted by large and small cations. Accordingly, modified zeolites with sodium ions may be preferred in some embodiments.

TABLE 4

| | Lactic Acid | Selectivity (mole %) | |
|---|---|---|---|
| Catalyst | Conversion % | Acetaldehyde | Acrylic Acid |
| Cs³/K-L-type zeolite | 57.9 | 22.6 | 24.2 |
| Na³/K-L-type zeolite | 70.6 | 27.1 | 36.3 |
| Li /K-L-type zeolite | 72.9 | 51.8 | 29 |

Dehydroxylation of Lactic Acid in a Water Solvent—Impregnation Amount(1).

A starting composition of 40% lactic acid by weight of water was reacted in the vapor phase with a variety of catalysts listed in Table 5 at 330° C., according to Reaction Protocol III above.

As shown in Table 5, gradually increasing in the amount of the Na₂HPO₄ supported on the base K-L zeolite system improves both the conversion and the selectivity while reducing the byproducts acetaldehyde and propionic acid. For this catalyst system, 7.1% loading appears to be in the optimal range for Na₂HPO₄ impregnation.

TABLE 5

| | | Selectivity (mole %) | | |
|---|---|---|---|---|
| | Lactic Acid | Acetal- | Propionic | Acrylic |
| Catalyst | Conversion % | dehyde | Acid | Acid |
| K-L-type zeolite | 80.1 | 19.7 | 8.4 | 33.5 |
| 2.1% Na₂HPO₄/K-L-type | 81.2 | 14.9 | 6.6 | 32.3 |
| zeolite | | | | |
| 3.6% Na₂HPO₄/K-L-type | 81 | 12.6 | 4.2 | 31.1 |
| zeolite | | | | |
| 7.1% Na₂HPO₄/K-L-type | 82.3 | 11 | 5.0 | 31.9 |
| zeolite | | | | |
| 14.0% Na₂HPO₄/K-L- | 79.9 | 15.3 | 5.3 | 32.2 |
| type zeolite | | | | |

Dehydroxylation of Lactic Acid in a Water Solvent—Impregnation Amount(2).

A starting composition of 40% D,L-lactic acid by weight of water was reacted in the vapor phase with a variety of catalysts listed in Table 6 at 340° C., according to Reaction Protocol III above.

As shown in Table 6, impregnation appears to yield a catalyst system with higher conversion rates. However, the amount of the Na₂HPO₄ supported on the base Na³/K-L zeolite system appears to effect the conversion and the selectivity while significantly reducing the byproduct acetaldehyde. For this catalyst system, about 7.1% loading appears to be in the optimal range for Na₂HPO₄ impregnation.

TABLE 6

| | Lactic | | | |
|---|---|---|---|---|
| | Acid | Selectivity (mole %) | | |
| | Conver- | Acetal- | Propionic | Acrylic |
| Catalyst | sion % | dehyde | Acid | Acid |
| Na³/K-L-type zeolite | 80.6 | 31.1 | 5.2 | 46.7 |
| 1.4% Na₂HPO₄/Na³/K-L- | 90.8 | 22.2 | 4.3 | 37.6 |
| type zeolite | | | | |
| 7.1% Na₂HPO₄/Na³/K-L- | 87.3 | 14.9 | 3.0 | 40.7 |
| type zeolite | | | | |
| 10.7% Na₂HPO₄/Na³/K- | 86.4 | 14.7 | 6.5 | 40.2 |
| L-type zeolite | | | | |
| 14% Na₂HPO₄/Na³/K-L- | 85.8 | 15.2 | 6.9 | 39.8 |
| type zeolite | | | | |

Dehydroxylation of Lactic Acid in a Water Solvent—Catalyst Stability.

A starting composition of 40% lactic acid/60% water was reacted in the vapor phase with a variety of catalysts listed in Table 7 at 340° C. for 4 hours, according to Reaction Protocol III above. As a measure of each catalyst's propensity to lose activity over time, the products were analyzed each hour of a four hour reaction.

As shown in Table 7, both the K-L zeolite and the Na³/K-L-type zeolite show gradually deteriorating activity and selectivity over time, although to a lesser extent with the Na³/K-L-type zeolite. In contrast, the impregnation with Na₂HPO₄ appears to stabilize both the conversion and the selectivity as shown for the 7.1% Na₂HPO₄/K-L-type zeolite and the 7.1% Na₂HPO₄/Na³/K-L-type zeolite catalysts. Both the partial Na exchange and Na₂HPO₄ impregnation (i.e., the 7.1% Na₂HPO₄/Na³/K-L-type zeolite) appears to have a synergistic effect that leads to a high and stable conversation at high and stable selectivity to acrylic acid.

TABLE 7

| | | Lactic | Selectivity (mole %) | | |
|---|---|---|---|---|---|
| | Time | Acid Con- | Acetal- | Propionic | Acrylic |
| Catalyst | (hr) | version % | dehyde | Acid | Acid |
| Tosoh K-L | 1 | 86.06 | 13.69 | 1.7 | 40.81 |
| | 2 | 79.80 | 16.70 | 2.2 | 39.69 |
| | 3 | 76.79 | 15.27 | 2.9 | 38.62 |
| | 4 | 76.19 | 12.31 | 3.0 | 32.98 |
| Na³/K-L- | 1 | 83.49 | 29.73 | 4.4 | 50.87 |
| type zeolite | 2 | 83.87 | 29.20 | 4.5 | 45.10 |
| | 3 | 77.89 | 32.73 | 5.6 | 46.65 |
| | 4 | 77.32 | 32.80 | 6.4 | 44.28 |
| 7.1% | 1 | 85.07 | 12.94 | 5.3 | 34.31 |
| Na₂HPO₄/K-L- | 2 | 83.42 | 14.52 | 6.8 | 36.89 |
| type zeolite | 3 | 82.78 | 14.38 | 7.2 | 35.94 |
| | 4 | 83.39 | 14.60 | 7.7 | 34.46 |
| 7.1% | 1 | 90.22 | 14.60 | 2.7 | 39.85 |
| Na₂HPO₄/Na³/K-L- | 2 | 87.58 | 14.58 | 2.9 | 41.64 |
| type zeolite | 3 | 86.56 | 15.27 | 3.0 | 40.94 |
| | 4 | 84.65 | 15.23 | 3.4 | 40.41 |

Dehydroxylation of Lactic Acid in a Water Solvent—Temperature Effect.

Starting compositions with varying concentrations of lactic acid (“LA”) in water as listed in Table 8 with 100 ppm of 4-methoxyphenol were reacted in the vapor phase with a 7.1% Na₂HPO₄/Na³/K-L-type zeolite, according to Reaction Protocol III above, for 4 hours at varying temperatures as listed in Table 8.

As shown in Table 8, for the 40% lactic acid starting composition, increasing the temperature led to increase in both the conversion and the selectivity. In contrast, at 30% and 20% lactic acid starting composition, increasing temperature appears to increase conversion but reduces selectivity towards acrylic acid, which was quite drastic when the temperature was increased to 340° C. for the sample having a 20% starting concentration of lactic acid.

TABLE 8

| | | Lactic | | | |
|---|---|---|---|---|---|
| Initial | Temper- | Acid | Selectivity (mole %) | | |
| Lactic Acid | ature | Conver- | Acetal- | Propionic | Acrylic |
| Concentration | (° C.) | sion % | dehyde | Acid | Acid |
| 20% | 320 | 78.6 | 13.8 | 8.0 | 43.7 |
| | 330 | 85.9 | 16.3 | 8.3 | 45.2 |
| | 340 | 92.5 | 15.3 | 13.4 | 32.3 |
| 30% | 320 | 73.5 | 15.5 | 8.0 | 41.7 |
| | 330 | 80.3 | 16.9 | 8.6 | 40.1 |
| | 340 | 89.0 | 17.7 | 11.3 | 40.2 |
| 40% | 320 | 67.3 | 10.8 | 4.1 | 30.5 |
| | 330 | 77.5 | 14.7 | 5.6 | 37.9 |
| | 340 | 87.3 | 14.9 | 3.0 | 40.7 |

Dehydroxylation of Lactic Acid in a Water Solvent—Lactic Acid Concentration Effect.

Starting compositions with 50% and 60% initial concentrations of lactic acid (“LA”) in water as listed in Table 9 were reacted in the vapor phase with a (7.1% Na₂HPO₄)/Na³/K-L-type zeolite, according to Reaction Protocol III above, for 5 hours at varying temperatures as listed in Table 9.

As shown in Table 9, increasing the temperature led to an increase in the conversion but the selectivity was minimally changed at the higher temperature. Further, increasing the initial lactic acid concentration has minimal effect, at least at these concentrations, on the conversion and selectivity at the corresponding temperatures.

TABLE 9

| Initial | | | Selectivity (mole %) | | |
|---|---|---|---|---|---|
| Lactic Acid | Temp | Lactic Acid | Acetal- | Propionic | Acrylic |
| Concentration | (° C.) | Conversion % | dehyde | Acid | Acid |
| 50% | 340 | 78.9 | 18.6 | 3.8 | 42.4 |
| | 350 | 86.7 | 22.0 | 5.5 | 47.3 |
| | 360 | 93.6 | 22.4 | 8.6 | 45.0 |
| 60% | 340 | 79.8 | 20.4 | 8.3 | 46.3 |
| | 350 | 85.4 | 23.5 | 8.3 | 47.9 |
| | 360 | 93.9 | 23.3 | 14.3 | 44.8 |

Dehydroxylation of Lactic Acid in a Water Solvent—Reactant Flow Rate Effect.

A starting composition of 40% lactic acid/60% water was reacted in the vapor phase with a Na³/K-L-type zeolite, according to Reaction Protocol III above, for 3 hours at 320° C., wherein the liquid flow rate of the starting composition was varied to account for the weight hourly space velocity (“WHSV”) in Table 10.

As shown in Table 10, increasing the lactic acid space velocity (i.e., increasing reactant flow rate) leads to reduction in the conversion and significant improvement in the acrylic acid selectivity.

TABLE 10

| | | Selectivity (mole %) | | |
|---|---|---|---|---|
| WHSV | Lactic Acid | | Propionic | Acrylic |
| (g_{X}/g_{C}/h) | Conversion % | Acetaldehyde | Acid | Acid |
| 0.600 | 70.6 | 27.1 | 0.0 | 36.3 |
| 1.200 | 54.1 | 32.9 | 0.0 | 49.9 |
| 1.500 | 50.9 | 33.0 | 0.0 | 52.8 |

Dehydroxylation of Lactic Acid in a Water Solvent—Carrier Gas Flow Rate Effect.

A starting composition of 40% lactic acid/60% water was reacted in the vapor phase with a Na³/K-L-type zeolite, according to Reaction Protocol III above, for 4 hours at 320° C., wherein the flow rate of the argon carrier gas was varied as listed in Table 11.

As shown in Table 11, increasing the carrier gas flow rate appears to reduce the acetaldehyde and propionic acid formation while maintaining acrylic acid selectivity.

TABLE 11

| Argon | | Selectivity (mole %) | | |
|---|---|---|---|---|
| Flow Rate | Lactic Acid | | Propionic | Acrylic |
| (mL/min) | Conversion % | Acetaldehyde | Acid | Acid |
| 5 | 70.6 | 27.1 | Not | 36.3 |
| | | | Measured | |
| 10 | 78.4 | 22.2 | 4.1 | 34.4 |
| 20 | 79.0 | 16.4 | 2.2 | 37.5 |
| 30 | 81.6 | 14.6 | 2.8 | 36.7 |
| 40 | 79.0 | 10.8 | 1.3 | 37.1 |

Dehydroxylation of Lactic Acid in a Solvent—Solvent Effect.

A starting composition as listed in Table 12 was reacted in the vapor phase with a Na³/K-L-type zeolite, according to Reaction Protocol III above, for 4 hours at 340° C.

As shown in Table 12, a Na³/K-L-type zeolite does catalyze esterification reactions. Further, the addition of an alcohol in the starting composition appears to have minimal effect on the conversion but does dramatically reduce the selectivity to acrylic acid.

TABLE 12

| | Lactic | Selectivity (mole %) | | | | | |
|---|---|---|---|---|---|---|---|
| Starting | Acid | Acetal- | | | | Acrylic | Propionic |
| Comp. | Conv. % | dehyde | Acrylate* | MOPAE*** | Lactate** | Acid | Acid |
| 30% LA 70% | 90.5 | 22.4 | 0 | 0 | 0 | 33.4 | 13.7 |
| Water | | | | | | | |
| 30% LA 65% | 92.2 | 14.9 | 1.6 | 1.0 | 20.8 | 23.0 | 6.8 |
| MeOH 5% | | | | | | | |
| Water | | | | | | | |
| 30% LA 65% | 93.5 | 15.9 | 0.4 | 0 | 10.1 | 20.2 | 5 |
| EtOH 5% | | | | | | | |
| Water | | | | | | | |

*methyl acrylate or ethyl acrylate for methanol (“MeOH”) and ethanol (“EtOH”), respectively.
**methyl lactate or ethyl lactate for methanol and ethanol, respectively.
***2-methoxypropionic acid methyl ester or 2-ethoxypropionic acid ethyl ester for methanol and ethanol, respectively.

Dehydroxylation of Lactic Acid in a Water Solvent—Catalyst Volume Effect.

A starting composition of 20% lactic acid/80% water was reacted in the vapor phase with a 7.1% Na₂HPO₄/Na³/K-L-type zeolite, according to Reaction Protocol III above, for 4 hours at 330° C., wherein the volume of the catalyst in the reactor was varied as listed in Table 13.

As shown in Table 13, increasing the amount of catalyst appears to increase the conversion. However, excess catalyst appears to decrease selectivity.

TABLE 13

| | | Selectivity (mole %) | | |
|---|---|---|---|---|
| Catalyst | Lactic Acid | | Propionic | Acrylic |
| (mL) | Conversion % | Acetaldehyde | Acid | Acid |
| 1 | 67.8 | 17.3 | 5.4 | 47.2 |
| 2 | 85.9 | 16.3 | 8.3 | 45.2 |
| 3 | 96.4 | 14.1 | 3.6 | 49.5 |
| 4 | 98.4 | 17.0 | 5.3 | 43.9 |
| 5 | 99.2 | 9.0 | 3.4 | 40.9 |
| 6 | 99.6 | 15.9 | 7.1 | 20.4 |
| 7 | 99.9 | 17.3 | 5.3 | 27.0 |

Dehydroxylation of Lactic Acid in a Water Solvent—Catalyst Activity Attrition.

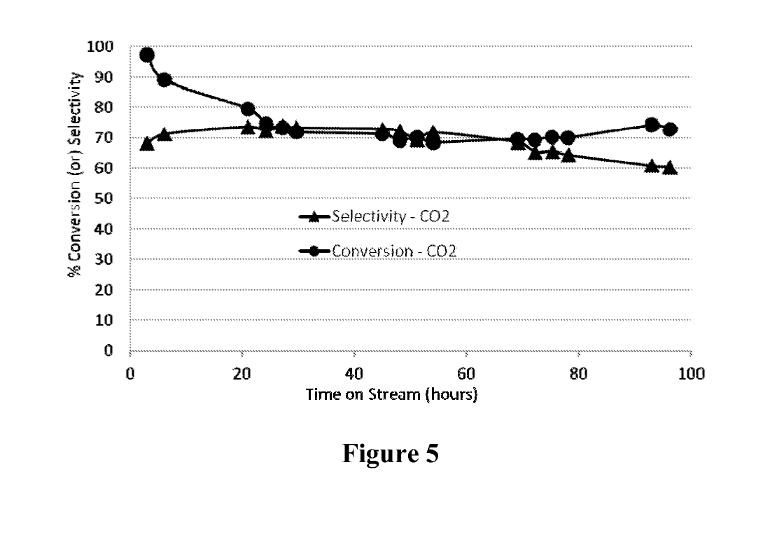

A starting composition of 20% lactic acid/80% water was reacted in the vapor phase over a 7.1% Na₂HPO₄/Na³/K-L-type zeolite at 330° C. for 96 hours, according to Reaction Protocol III above (FIG. 5). As a measure of each catalyst's propensity to lose activity over time, the products were each analyzed periodically throughout the 96-hour reaction.

As shown in FIG. 5, the 7.1% Na₂HPO₄/Na³/K-L-type zeolite has consistent lactic acid conversion and acrylic acid selectivity of about 70% or greater for over 50 hours.

Dehydroxylation of Lactic Acid in a Water Solvent—Lactic Acid Source.

Starting compositions with either 20% or 30% concentrations of lactic acid were reacted in the vapor phase with a 7.1% Na₂HPO₄/Na³/K-L-type zeolite, according to Reaction Protocol III above, for 4 hours at 330° C. The lactic acid was from one of two sources (1) synthetic lactic acid (available from Sigma-Aldrich, TCI, or Alfa Aesar), and (2) bio-derived lactic acid (available from ADM Corporation). The bio-derived lactic acid was derived from biological fermentation of sugars. Two different grades (USP—United States Pharmacopia; FCC—Food Chemicals Codex) of bio-derived lactic acid were used in this experiment (Table 14).

TABLE 14

| Initial | | | | | |
|---|---|---|---|---|---|
| Lactic | Lactic | Lactic | Selectivity (mole %) | | |
| Acid | Acid | Acid | Acetal- | Propionic | Acrylic |
| Conc. | Source | Conv. % | dehyde | Acid | Acid |
| 20% | synthetic | 85.9 | 16.3 | 8.3 | 45.2 |
| | bio-USP grade | 83.1 | 21.0 | 5.8 | 51.1 |
| | bio-FCC grade | 81.6 | 20.5 | 7.7 | 49.9 |
| 30% | synthetic | 80.3 | 16.9 | 8.6 | 40.1 |
| | bio-USP grade | 75.9 | 20.9 | 5.6 | 46.6 |
| | bio-FCC grade | 83.4 | 20.8 | 7.5 | 47.8 |

Dehydroxylation of Butyl Lactate in a Butanol-Water Solvent—Catalyst Effect.

A starting composition of 50% butyl lactate/45% butanol/5% water was reacted in the vapor phase with a variety of catalysts as listed in Table 15, according to Reaction Protocol II above, for 4 hours at 300° C.

As shown in Table 15, increasing the amount of Na⁺ associated with the catalyst (i.e., more Na⁺ exchanges) increases the selectivity to acrylic acid, an effect seen also in the Li⁺ exchanged catalyst.

TABLE 15

| | Butyl | | | | |
|---|---|---|---|---|---|
| | Lactate | Selectivity (mole %) | | | |
| | Conver- | Acetal- | Butyl | Propionic | Acrylic |
| Catalyst | sion % | dehyde | Acrylate | Acid | Acid |
| K-L-type | 42.6 | 5.9 | 1.5 | 2.4 | 16.7 |
| zeolite | | | | | |
| Na/K-L-type | 49.3 | 10.8 | 1.9 | 3.4 | 30.3 |
| zeolite | | | | | |
| Na²/K-L-type | 45.8 | 15.0 | 2.0 | 5.3 | 39.2 |
| zeolite | | | | | |
| Na³/K-L-type | 53.2 | 17.5 | 1.9 | 3.7 | 42.9 |
| zeolite | | | | | |
| Na⁴/K-L-type | 54.0 | 14.2 | 1.8 | 3.4 | 38.4 |
| zeolite | | | | | |

Dehydroxylation of Butyl Lactate in a Butanol-Water Solvent—Water Effect.

A starting composition as listed in Table 16 was reacted in the vapor phase with a Na³/K-L-type zeolite, according to Reaction Protocol II above, for 4 hours at 300° C.

As shown in Table 16, in solvent systems that include water, increasing the water content increases the acrylic acid selectivity. Accordingly, for these reaction parameters, an optimal range of water content may be around about 5 to about 10% resulting in stabilization of catalyst activity at the temperature tested.

TABLE 16

| | Butyl | | | | |
|---|---|---|---|---|---|
| | Lactate | Selectivity (mole %) | | | |
| Starting | Conver- | Acetal- | Butyl | Propionic | Acrylic |
| Composition | sion % | dehyde | Acrylate | Acid | Acid |
| 50% butyl lactate | 39.3 | 16.4 | 2.5 | 4.8 | 37.6 |
| 50% butanol 0% | | | | | |
| water | | | | | |
| 50% butyl lactate | 45.7 | 12.5 | 1.7 | 3.6 | 29.1 |
| 49% butanol 1% | | | | | |
| water | | | | | |
| 50% butyl lactate | 55.0 | 15.8 | 2.1 | 3.6 | 38.5 |
| 47.5% butanol 2.5% | | | | | |
| water | | | | | |
| 50% butyl lactate | 55.9 | 12.0 | 1.5 | 2.8 | 31.7 |
| 46.5% butanol 3.5% | | | | | |
| water | | | | | |
| 50% butyl lactate | 53.2 | 17.5 | 1.9 | 3.7 | 42.9 |
| 45% butanol 5% | | | | | |
| water | | | | | |
| 50% butyl lactate | 51.0 | 17.0 | 1.9 | 2.0 | 44.3 |
| 40% butanol 10% | | | | | |
| water | | | | | |

Dehydroxylation of Butyl Lactate in a Butanol-Water Solvent—Temperature Effect.

A starting composition of 50% butyl lactate/45% butanol/5% water was reacted in the vapor phase with Na³/K-L-type zeolite, according to Reaction Protocol II above, for 4 hours at a temperature as listed in Table 17.

As shown in Table 17, increasing the temperature above 280° C. appears to increase the butyl lactate conversion and acrylic acid selectivity.

TABLE 17

| | | Selectivity (mole %) | | | |
|---|---|---|---|---|---|
| Temp | Butyl Lactate | | Butyl | Propionic | Acrylic |
| (° C.) | Conversion % | Acetaldehyde | Acrylate | Acid | Acid |
| 280 | 32.8 | 10.1 | 0 | 3.0 | 24.6 |
| 290 | 33.6 | 10.8 | 0 | 2.5 | 28.3 |
| 300 | 53.2 | 17.5 | 1.9 | 3.7 | 42.9 |
| 310 | 49.3 | 17.0 | 3.2 | 4.6 | 49.7 |
| 320 | 50.7 | 18.2 | 3.6 | 4.8 | 52.5 |

Dehydroxylation of Butyl Lactate in a Butanol-Water Solvent—Reactant Flow Rate Effect.

A starting composition of 50% butyl lactate/45% butanol/5% water was reacted in the vapor phase with Na³/K-L-type zeolite, according to Reaction Protocol II above, at 300° C. for 4 hours. The flow rate of the starting composition was adjusted to yield the LHSV as listed in Table 18.

As shown in Table 18, increasing the LHSV appears to have a minimal effect on the acrylic acid selectivity but the butyl lactate conversion has dropped. However, an optimal LHSV for acrylic acid selectivity appears to be about 2 hr⁻¹ to about 3 hr⁻¹.

TABLE 18

| | | Selectivity (mole %) | | | |
|---|---|---|---|---|---|
| LHSV | Butyl Lactate | Acetal- | Butyl | Propionic | Acrylic |
| (h⁻¹) | Conversion % | dehyde | Acrylate | Acid | Acid |
| 1.5 | 41.8 | 18.7 | 3.2 | 4.8 | 57.9 |
| 2 | 34.7 | 19.7 | 2.6 | 5.0 | 60.1 |
| 3 | 25.9 | 19.9 | 1.9 | 4.9 | 59.3 |
| 6 | 14.5 | 19.4 | 0 | 0 | 56.9 |

Dehydroxylation of Lactic Acid in a Water Solvent—Catalytic Activity After Regeneration.

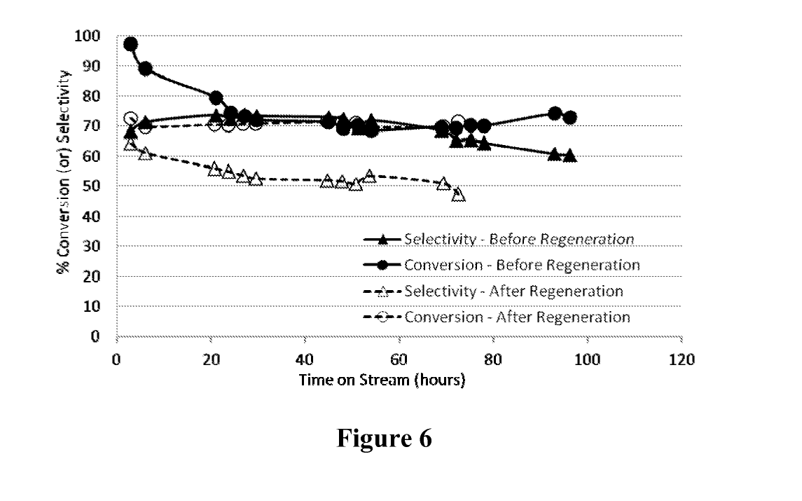

A starting composition of 20% lactic acid/80% water was reacted in the vapor phase over a 7.1% Na₂HPO₄/Na³/K-L-type zeolite at 330° C. for 96 hours, according to Reaction Protocol III above (FIG. 6), and regenerated with air for 3 hours at 330° C. As a measure of catalyst's propensity for activity after regeneration, the products were each analyzed periodically throughout the 72-hour reaction. As shown in FIG. 6, the steady state conversion of 70% or greater was successfully regained. However, due to the lower temperature used for regeneration than required to burn the deposited coke because of equipment limitation, the steady state acrylic acid selectivity was reduced to about 50% or greater.

Dehydroxylation of Methyl-Lactate in a Methanol-Water Solvent.

A starting composition as listed in Table 19 was reacted in the vapor phase with Na³/K-L-type zeolite, according to Reaction Protocol I above, at 300° C. for 4 hours.

As shown in Table 19, at increasing water concentrations, the acrylic acid selectivity increases. Also, the byproduct methoxy propionic acid was reduced with increasing water concentrations.

TABLE 19

| | | Selectivity (mole %) | | | |
|---|---|---|---|---|---|
| | Methyl | | | Methoxy | |
| Starting | Lactate | Acetal- | Methyl | Propionic | Acrylic |
| Composition | Conv. % | dehyde | Acrylate | Acid | Acid |
| 50% methyl | 41.3 | 13.7 | 16.2 | 12.7 | 20.5 |
| lactate 45% | | | | | |
| methanol 5% | | | | | |
| water | | | | | |
| 50% methyl | 40.6 | 14.2 | 18.0 | 13.8 | 28.8 |
| lactate 40% | | | | | |
| methanol 10% | | | | | |
| water | | | | | |
| 50% methyl | 39.8 | 14.8 | 12.8 | 7.8 | 35.3 |
| lactate 30% | | | | | |
| methanol 20% | | | | | |
| water | | | | | |

Dehydroxylation of Lactic Acid in a Water Solvent—Catalytic Activity with a Binder.

Starting compositions with 20% concentrations of lactic acid were reacted in the vapor phase with a calcined or un-calcined catalyst listed in Table 20, according to Reaction Protocol III above, for 5 hours at 330° C. Catalyst HSZ-500™ KOD1C (a L-type zeolite, available from TOSOH USA, Inc.) was either used as is or calcined at 450° C. Clay-A was used as a binder in this experiment. The Clay-A binder used in this experiment is different from the silica binder in all other experiments described in this patent application.

The results shown in Table 20 indicate the incorporation of a calcined catalyst versus an un-calcined catalyst with a Clay-A may minimally affect the conversion rate but may act to decrease the acrylic acid selectivity and increase byproduct acetaldehyde. This could be due to the modification of the acidic or basic sites on the catalyst during calcination process.

TABLE 20

| | | Selectivity (mole %) | | |
|---|---|---|---|---|
| | Lactic Acid | | Propionic | Acrylic |
| Catalyst | Conversion % | Acetaldehyde | Acid | Acid |
| un-calcined | 80.1 | 23.8 | 9.8 | 36.0 |
| calcined | 84.8 | 33.0 | 4.4 | 32.4 |

Dehydroxylation of Lactic Acid in a Water Solvent—Effect of Carrier Gas Composition.

Starting compositions with 20% concentrations of lactic acid were reacted in the vapor phase using a carrier gas as listed in Table 21 with a variety of catalysts as listed in Table 21, according to Reaction Protocol III above, for 5 hours at 330° C.

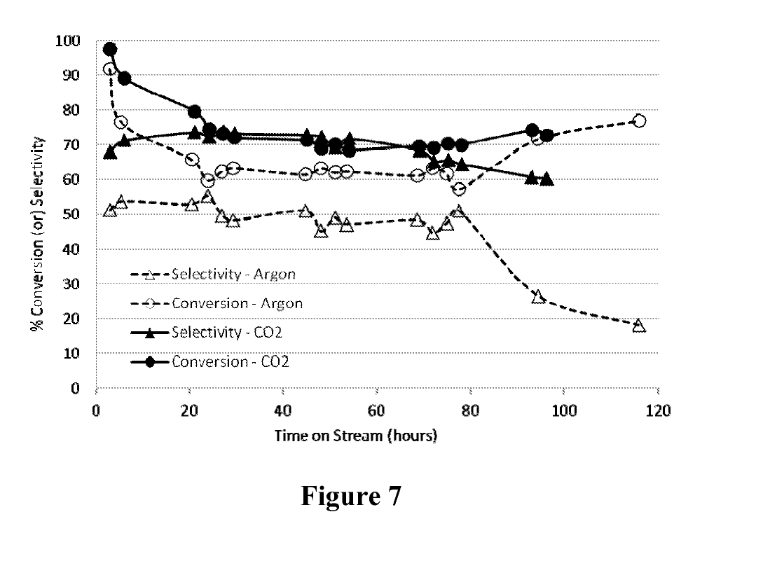

The results shown in Table 21 indicate that using carbon dioxide as a carrier gas improves both the stable conversion percentage and the acrylic acid selectivity with both modified L-type zeolite catalysts tested reducing the byproduct acetaldehyde formation in particular for 7.1% Na₂HPO₄/Na-3^{rd} Ex K-L. FIG. 7 presents the comparison data on the 20% aqueous lactic acid conversion and acrylic acid selectivity for 7.1% Na2HPO4/Na-3^{rd} Ex K-L using CO₂ and argon or nitrogen as carrier gases during the course of 100 hours.

TABLE 21

| | | Lactic | Selectivity (mole %) | | |
|---|---|---|---|---|---|
| Carrier | | Acid | A_{c}etal- | Propionic | Acrylic |
| Gas | Catalyst | Conv. % | dehyde | Acid | Acid |
| Argon | Na-3^{rd} | 91.5 | 35.8 | 7.0 | 50.2 |
| | exchanged K-L | | | | |
| | 7.1%Na₂HPO₄/Na- | 80.2 | 17.4 | 11.2 | 51.8 |
| | 3^{rd} Ex K-L | | | | |
| CO² | Na-3^{rd} | 92.3 | 29.4 | 9.1 | 49.3 |
| | exchanged K-L | | | | |
| | 7.1%Na₂HPO₄/Na- | 85.5 | 16.8 | 6.0 | 59.1 |
| | 3^{rd} Ex K-L | | | | |

Measurement of Corrosion of Stainless Steel Reactor Walls.

In this experiment, the extent of the corrosion of the stainless steel reactor wall by lactic acid was monitored during the course of four days of continuous operation of stainless steel catalytic reactor with L-type zeolite catalyst. Aqueous lactic acid solution (20% w/v) was used as feed. The reactor was maintained at the temperature of 330° C. and nitrogen was used as a carrier gas. The reactor was operated in a continuous mode and acrylic acid was collected on a daily basis. At the end of the fourth day, the acrylic acid fractions collected on a daily basis, the lactic acid feed, fresh catalyst, and the spent catalyst were analyzed for the presence of levels of each metal in each of these samples, shown in Table 22. The results indicate that the L-type zeolite catalyst retains significant concentrations of metals leached from the stainless reactor in the dehydroxylation reaction.

TABLE 22

| | Quantity (mg/kg) | | | | | | |
|---|---|---|---|---|---|---|---|
| | Lactic | Acrylic | Acrylic | A^{c}rylic | Acrylic | Fresh | Spent |
| | acid | Acid | Acid | Acid | Acid | Cata- | Cata- |
| Metal | feed | Day 1 | Day 1 | Day 3 | Day 4 | lyst | lyst |
| Iron | 0.3 | 131.0 | 113.4 | 82.4 | 4.2 | 144 | 24565 |
| (Fe) | | | | | | | |
| Nickel | — | 15.4 | 12.9 | 10.5 | 1.2 | 2 | 4317 |
| (NI) | | | | | | | |
| Chro- | — | 33.7 | 30.4 | 0.9 | 0.9 | 2 | 6850 |
| mium | | | | | | | |
| (Cr) | | | | | | | |

In this lactic acid dehydroxylation experiment, acetaldehyde and propionic acid were also detected as byproducts and their mole selectivity was also determined during the course of the four days of experimentation. As the results shown Table 23 indicate, the mole selectivity for acetaldehyde and propionic acid increased during the course of four days, thereby decreasing the selectivity for acrylic acid.

TABLE 23

% Mole selectivity

| Product | Day 1 | Day 2 | Day 3 | Day 4 |
|---|---|---|---|---|
| Acetaldehye | 11.3 | 10.56 | 11.61 | 14.4 |
| Propionic acid | 4 | 5.8 | 7.9 | 22.9 |

The examples above illustrate that a plurality of catalysts based on L-type zeolites and modified zeolites can be used for the production of α,β-unsaturated carboxylic acids and/or esters thereof.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A method comprising:
providing a composition comprising a reactant selected from the group consisting of an α-hydroxycarboxylic acid, an α-hydroxycarboxylic acid ester, a β-hydroxycarboxylic acid, a β-hydroxycarboxylic acid ester, an α-alkoxycarboxylic acid, an α-alkoxycarboxylic acid ester, a β-alkoxycarboxylic acid, a β-alkoxycarboxylic acid ester, a lactide, and any combination thereof; and
performing a dehydroxylation reaction by contacting the composition with a dehydroxylation catalyst, thereby producing a product comprising an α,β-unsaturated carboxylic acid and/or ester thereof, the dehydroxylation catalyst comprising at least one selected from the group consisting of an L-type zeolite, a modified L-type zeolite, and any combination thereof.

2. The method of claim 1 further comprising:
performing an esterification reaction by contacting the product with an esterification catalyst and an alcohol, thereby producing a second product comprising an α,β-unsaturated carboxylic acid ester.

3. The method of claim 2, wherein the dehydroxylation catalyst and the esterification catalyst are the same.

4. The method of claim 2, wherein the dehydroxylation reaction and the esterification reaction are performed concurrently.

5. The method of claim 2, wherein the dehydroxylation reaction and the esterification reaction are performed in series.

6. The method of claim 2, wherein the dehydroxylation reaction and the esterification reaction are performed in a single reactor vessel.

7. The method of claim 2, wherein the esterification reaction is performed in a reactor vessel comprising a reactor material comprising at least one selected from the group consisting of titanium, silanized stainless steel, quartz, and any combination thereof.

8. The method of claim 2, wherein the alcohol is at least one selected from the group consisting of C₁-C₂₀ alcohol, an aryl alcohol, a cyclic alcohol, and any combination thereof.

9. The method of claim 2, wherein the alcohol is at least one selected from the group consisting of methanol, ethanol, propanol, iso-propanol, n-propanol, butanol, iso-butanol, n-butanol, 2-ethylhexanol, iso-nonanol, iso-decylalcohol, 3-propylheptanol, benzyl alcohol, cyclohexanol, cyclopentanol, and any combination thereof.

10. The method of claim 2, wherein the esterification reaction occurs in the presence of a carrier gas that comprises greater than about 90% carbon dioxide.

11. The method of claim 1, wherein the dehydroxylation reaction is performed in a reactor vessel comprising a reactor material comprising at least one selected from the group consisting of titanium, silanized stainless steel, quartz, and any combination thereof.

12. (canceled)

13. The method of claim 1, wherein the dehydroxylation reaction occurs in the presence of a carrier gas that comprises greater than about 90% carbon dioxide.

14. The method of claim 1, wherein the reactants are in the vapor phase and the dehydroxylation catalyst is in the solid phase.

15. The method of claim 1, wherein the dehydroxylation catalyst further comprises at least one additional component selected from the group consisting of a solid oxide, a zeolite other than the L-type zeolite, an acid catalyst, a weak acid catalyst, a strong acid catalyst, a neutral catalyst, a basic catalyst, and any combination thereof.

16. The method of claim 1, wherein the modified L-type zeolite comprises at least one inorganic salt that comprises at least one ion selected from the group consisting or a phosphate, a sulfate, a molybdate, a tungstate, a stagnate, an animonate, and any combination thereof.

17. The method of claim 16, wherein the inorganic salt is present in the modified L-type zeolite at a concentration of about 0.1 mmol/g modified zeolite to about 1.0 mmol/g modified zeolite.

18. The method of claim 16, wherein the inorganic salt comprises at least one selected from the group consisting of monosodium phosphate, disodium phosphate, and trisodium phosphate, a potassium phosphate, a sodium aluminum phosphate compound, and any combination thereof.

19. The method of claim 1, wherein the modified L-type zeolite has undergone at least one ion exchange.

20. The method of claim 1, wherein the modified L-type zeolite has associated therewith at least one ion elected from the group consisting of H⁺, Li⁺, Na⁺, K⁺, Cs⁺, Mg²⁺, Ca²⁺, La²⁺, La³⁺, Ce²⁺, Ce³⁺, Ce⁴⁺, Sm²⁺, Sm³⁺, Eu²⁺, Eu³⁺, and any combination thereof.

21. The method of claim 1, wherein the modified L-type zeolite is a Na/K-L-type zeolite having a ratio of sodium ions to potassium ions of about 1:10 or greater.

22. The method of claim 1, wherein the dehydroxylation catalyst has conversion efficiency of about 75% or greater.

23. The method of claim 1, wherein the dehydroxylation catalyst has conversion efficiency of about 90% or greater.

24. The method of claim 1, wherein the product comprises about 60 mole % or greater of the α,β-unsaturated carboxylic acid and/or ester thereof.

25. The method of claim 1, wherein the reactant is lactide.

26. The method of claim 1, wherein the reactant is biologically-derived.

27. The method of claim 1, wherein the dehydroxylation reaction is performed in the presence of a polymerization inhibitor.

28. The method of claim 1, wherein the composition further comprises a solvent.

29. The method of claim 28 further comprising:
recycling the solvent after the dehydroxylation reaction.

30. A method comprising:
providing a composition comprising a reactant selected from the group consisting of an α-hydroxycarboxylic acid, a β-hydroxycarboxylic acid, an α-alkoxycarboxylic acid, a β-alkoxycarboxylic acid, and any combination thereof;
performing an esterification reaction by contacting the composition with an esterification catalyst and an alcohol, thereby producing an intermediate comprising an ester of the reactant; and
then performing a dehydroxylation reaction by contacting intermediate with a dehydroxylation catalyst, thereby producing a product comprising an α,β-unsaturated carboxylic acid ester, the dehydroxylation catalyst comprising at least one selected from the group consisting of an L-type zeolite, a modified L-type zeolite, and any combination thereof.

31. The method of claim 30, wherein the dehydroxylation reaction and/or the esterification reaction occur in the presence of a carrier gas that substantially comprises carbon dioxide.

32. The method of claim 30, wherein the dehydroxylation reaction and/or the esterification reaction are performed in a reactor vessel comprising a reactor material comprising at least one selected from the group consisting of titanium, silanized stainless steel, quartz, and any combination thereof.

33. The method of claim 30, wherein the dehydroxylation catalyst and the esterification catalyst are the same.

34. The method claim 30, wherein the alcohol is at least one selected from the group consisting of a C₁-C₂₀ alcohol, an aryl alcohol, acyclic alcohol, and any combination thereof.

35. The method of claim 30, wherein the alcohol is at least one selected from the group consisting of methanol, ethanol, propanol, iso-propanol, n-propanol, butanol, iso-butanol, n-butanol, 2-ethylhexanol, iso-nonanol, iso-decylalcohol, 3-propylheptanol, benzyl alcohol, cyclohexanol, cyclopentanol, and any combination thereof.

36. The method of claim 30, wherein the modified L-type zeolite comprises at least one inorganic salt that comprises at least one ion selected from the group consisting of a phosphate, a sulfate, a molybdate, a tungstate, a stagnate, an antimonite, and any combination thereof.

37. The method of claim 30, wherein the inorganic salt is present in the modified L-type zeolite at a concentration of about 0.1 mmol/g modified zeolite to about 1.0 mmol/g modified zeolite.

38. The method of claim 30, wherein the inorganic salt comprises at least one selected form the group consisting of monosodium phosphate, disodium phosphate, and trisodium phosphate, a potassium phosphate, a sodium aluminum phosphate compound, and any combination thereof.

39. The method of claim 30, wherein the modified L-type zeolite has undergone at least one ion exchange.

40. The method of claim 30, wherein the modified L-type zeolite has associated therewith at least one selected from the group consisting of H⁺, Li⁺, Na⁺, K⁺, Cs⁺, Mg²⁺, Ca²⁺, La²⁺, La³⁺, Ce²⁺, Ce³⁺, Ce⁴⁺, Sm²⁺, Eu²⁺, Eu³⁺, and any combination thereof.

41. The method of claim 30, wherein the modified L-type zeolite is a Na/K-L-type zeolite having a ratio of sodium ion to potassium ions of about 1:10 or greater.

42. The method of claim 30, wherein the dehydroxylation catalyst has conversion efficiency of about 75% or greater.

43. The method of claim 30, wherein the dedhydroxylation catalyst has conversion efficiency of about 90% or greater.

44. The method of claim 30, wherein the product comprises about 60 mole % or greater of the α,β-unsaturated carboxylic acid ester.

45. The method of claim 30, wherein the dehydroxylation reaction is performed in the presence of a polymerization inhibitor.

46. The method of claim 30, wherein the reactant is biologically-derived.

47. The method of claim 30, wherein the composition further comprises a solvent.

48. The method of claim 47 further comprising:
recycling solvent after the dehydroxylation reaction.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A method for manufacturing an acrylic acid or an acrylic acid ester from glycerol, the method comprising the steps of:
(a) catalytic conversion of the glycerol into a lactic acid or an alkyl lactate; and
(b) catalytic conversion of the lactic acid or the alkyl lactate from step (a) into acrylic acid or acrylic esters using a dehydroxylation catalyst comprising at least one selected from the group consisting of an L-type zeolite, a modified L-type zeolite, and any combination thereof.

64. The method of claim 63 further comprising purifying acrylic acid/acrylic ester from step (b)

65. (canceled)

66. (canceled)

67. (canceled)

68. The method of claim 63, wherein the modified L-type zeolite comprises at least one inorganic salt selected from the group consisting of a phosphate, a sulfate, a molybdate, a tungstate, a stanate, an antimonate, and any combination thereof.

69. The method of claim 63, wherein the modified L-type zeolite has undergone at least one ion exchange.

70. The method of claim 69, wherein the modified L-type zeolite has associated therewith at least one ion selected from the group consisting of H⁺, Li⁺, Na⁺, K⁺, Cs⁺, Mg²⁺, Ca²⁺, La²⁺, La³⁺, Ce²⁺, Ce³⁺, Ce⁴⁺, Sm²⁺, Sm³⁺, Eu²⁺, Eu³⁺, and any combination thereof.

71. The method of claim 63, wherein the dehydroxylation catalyst further comprises at least one selected from the group consisting of a solid oxide, a zeolite other than the L-type zeolite, an acid catalyst, a weak acid catalyst, a strong acid catalyst, a neutral catalyst, a basic catalyst, and any combination thereof.

72. (canceled)

73. (canceled)
